(19) United States
(12) Patent Application Publication
(10) Pub. No.: US 2018/0193294 A1
(43) Pub. Date: Jul. 12, 2018

(54) Dehydroabietic acid (DHAA) derivatives for use as ion channel openers

(71) Applicants: Fredrik ELINDER, Hässelby (SE); Peter KONRADSSON, Skänninge (SE); Sara LIIN, Linköping (SE); Nina OTTOSSON, Linghem (SE); Wu XIONGYU, Linköping (SE)

(72) Inventors: Fredrik ELINDER, Hässelby (SE); Peter KONRADSSON, Skänninge (SE); Sara LIIN, Linköping (SE); Nina OTTOSSON, Linghem (SE); Wu XIONGYU, Linköping (SE)

(21) Appl. No.: 15/646,296

(22) Filed: Jul. 11, 2017

Related U.S. Application Data
(63) Continuation of application No. PCT/SE2016/050008, filed on Jan. 11, 2016.

(30) Foreign Application Priority Data
Jan. 12, 2015 (SE) 1550018-4
Feb. 4, 2015 (SE) 1550118-2

(51) Int. Cl. A61K 31/192; A61P 25/08

(52) U.S. Cl. CPC A61K 31/192; A61P 25/08

(57) ABSTRACT

The present invention relates to derivatives of dehydroabietic acid useful in treatment of cardiac arrhythmia or a hyperexcitablity disease, such as epilepsy or pain, by extracellularly acting on the voltage sensitive domain (VSD) to open at least one member of the family of voltage-gated Kv (potassium) channels.

### BACKGROUND OF THE INVENTION

Voltage-gated ion channels play vital roles in generating cellular excitability, causing diseases when mutated, and being the target for drugs against diseases with increased cellular excitability, such as epilepsy, cardiac arrhythmia, and pain. Despite long-term efforts to develop effective medical drugs, many patients do not respond satisfactorily to the present-day drugs. For instance, one third of the patients with epilepsy do not respond properly. Therefore there is a need for new treatments. Voltage-gated ion channels, responsible for the generation and propagation of nervous and cardiac action potentials, are obvious targets.

Voltage-gated ion channels have a common structure: Four subunits packed together around an ion conducting pore. Each subunit has 6 transmembrane segments, named S1 to S6. The pore domain (S5-S6) includes the ion-conducting pore with the selectivity filter and the gates that open and close the pore. The voltage-sensor domain (VSD, S1-S4) includes the positively charged voltage sensor S4 which moves through the channel protein during activation of the channel. Many of the present-day drugs block voltage-gated ion channels by plugging the ion-conducting pore. In most cases Na channels are targeted, but also Ca and K channels. However, there is an alternative mechanism that potentially can affect ion channel conductance—instead of blocking the ion conducting pore, a drug can affect (i) the gate that open and close the channel, or (ii) the voltage sensor that affects the gate. Retigabine, a new antiepileptic drug, opens the M-type K channel by acting on the gate and consequently shutting down electrical excitability. Spider toxins and some other compounds have been shown to specifically act on the voltage-sensor domain (VSD) of the ion channel, but there is presently no medical drug acting on the VSD.

Many relatively common diseases such as epilepsy, cardiac arrhythmia, and chronic pain, depend on an increased electrical excitability.

A mechanism has been described, whereby charged hydrophobic compounds bind close to the VSD and thereby electrostatically affect the charged voltage sensor in the VSD. Negatively charged lipophilic substances (e.g. polyunsaturated fatty acids, PUFAs) was disclosed to bind to the lipid bilayer close to the ion channel and thereby shifting the channel's voltage dependence by electrostatically affecting the channel's voltage sensor, see Börjesson, S. I., et al Biophys. J. 95, 2242-2253 (2008). The binding site of PUFA is at the extracellular end of S3 and S4, distinct from previously described binding sites, and it is mainly the final opening step of the channel that is affected, see Börjesson, S. I. & Elinder, F., J. Gen. Physiol. 137, 563-640 (2011). However, to develop drug-like small-molecule compounds acting on voltage-gated channels with beneficial effects on for example epilepsy and pain, there is a need for other molecules than PUFAs.

A K channel is made supersensitive to PUFAs by inserting two extra positively charged residues in the extracellular end of the voltage sensor S4 (the 3R mutation), see. Ottosson, N. E. et al. J. Gen. Physiol. 143, 173-182 (2014). During certain circumstances, this channel increases the gain in open probability caused by PUFAs by more than 500 times compared to wild type. It was also described in this article that a resin acid, pimaric acid, had similar effects as PUFAs on the channel's voltage dependence.

Y-M Cui et al. Bioorg Med Chem, 18, 8642-8659 (2010) discloses that pimaric acid and other diterpene analogues, such as abietic acid and derivatives thereof have activity to open the calcium-activated BK channels, a subtype of K channels.

It is evident that there is a need for small-molecule drug candidates with potent properties to electrostatically open K channels and thereby being candidates to treat cardiac arrhythmia, epilepsy, and pain by compounds acting extracellularly on the voltage-sensor domain, rather than the traditional target, the ion-conducting pore domain.

The present patent application therefore is directed to derivatives of dehydroabietic acid that are demonstrated as potent openers of a specific voltage-gated K channel and thereby can be developed into drugs against cardiac arrhythmia and other hyperexcitability diseases including epilepsy and pain.

### DESCRIPTION AND SUMMARY OF THE INVENTION

The present invention relates to dehydroabietic acid derivatives according to formula I and all stereoisomers thereof, wherein R₁₁, R₁₂, and R₁₄ are independently selected from hydrogen, halogen, and R₂; R₁₃ is selected from hydrogen, halogen and R₃; and R₇ is selected from hydrogen, halogen, hydroxyl (—OH), carbonyl (═O), and ═N—O—R₁; where R₁ is selected from hydrogen, and saturated or unsaturated lower alkyl groups (C1-C6 alkyl, C2-C6 alkenyl); R₂ and R₃ are independently from each other selected from straight, branched or cyclic saturated or unsaturated hydrocarbons comprising from 1 to 6 carbon atoms (C1-C6 alkyl, C2-C6 alkenyl and C3-C6 cycloalkyl), for use in treatment of cardiac arrhythmia, or a hyperexcitability disease, by extracellularly acting on the voltage-sensor domain (VSD) to open at least one member of the family of voltage-gated potassium (Kv) channels (Kv family), wherein formula I is:

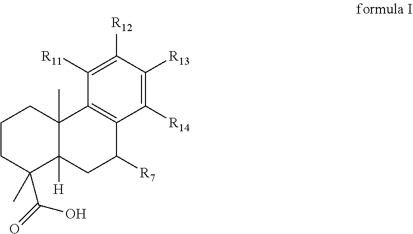

In the context of the present invention the definition dehydroabietic acid derivatives according to formula I and all stereoisomers thereof would, for example therefore encompass also variants of chirality of the carboxylic acid. For this reason, both the compounds (1R,4aS)-8-chloro-7-isopropyl-1,4a-dimethyl-1,2,3,4,4a,9,10,10a-octahydrophenanthrene-1-carboxylic acid and (1 S,4aS)-6-chloro-1,4a-dimethyl-1,2,3,4,4a,9,10,10a-octahydrophenanthrene-1-carboxylic acid should be covered by formula I be regarded within the scope of invention, even if the compound (1S,4aS)-6-chloro-1,4a-dimethyl-1,2,3,4,4a,9,10,10a-octahydrophenanthrene-1-carboxylic acid also can be termed a derivative of podocarpic acid. For this reason the term “dehydroabietic acid derivatives according to formula I and all stereoisomers thereof” should also be regarded to include derivatives of podocarpic acid.

Further in the context of the present invention, the groups R₂ and R₃ when defined as C1-C6 alkyl, C2-C6 alkenyl and C3-C6 cycloalkyl, as possible substitutes in positions R₁₁, R₁₂, R₁₄ and R₁₃ of formula I, wherein hydrogens of said alkyl, alkenyl, and cycloalkyl groups optionally can be substituted with at least one halogen.

According to one aspect of the invention the voltage-gated Kv (potassium) channel of the Kv family is selected from at least one of the subfamilies Kv-1, Kv-2, Kv-3, Kv-4, and Kv-7.

According to another aspect of the invention the voltage-gated potassium (Kv) channel of the Kv family is the subfamily Kv1.

In one aspect the previously defined dehydroabietic acid derivatives are for use in treatment of epilepsy or pain. At least one dehydroabietic acid derivative according to the invention is administered in a therapeutically active amount in a pharmaceutical dose form.

In one aspect the previously defined dehydroabietic acid derivatives are for use in treatment cardiac arrhythmia, especially atrial fibrillation (AF) in cardiac arrhythmia. At least one dehydroabietic acid derivative according to the invention is administered in a therapeutically active amount in a pharmaceutical dose form.

In one aspect the previously defined dehydroabietic acid derivatives are used as defined above and R₃ is an isopropyl group. In this aspect, R₁₃ preferably is isopropyl.

In one aspect, the dehydroabietic acid according to the invention are used as defined above and selected from compounds included in the groups a) to m):
a) dehydroabietic acid derivatives according to formula I and all stereoisomers thereof, wherein R₁₂ is —F, R₁₁ and R₁₄ is —H, R₃ is isopropyl, and R₇ is selected from —H, ═O and ═N—O—R₁, where R₁ is selected from —H, —CH₃, and —CH₂—CH═CH₂;
b) dehydroabietic acid derivatives according to formula I and all stereoisomers thereof, wherein R₁₁ and R₁₂ is —H, R₁₄ is —F, R₃ is isopropyl, and R₇ is selected from —H, ═O and ═N—O—R₁, where R₁ is selected from —H, —CH₃, and —CH₂—CH═CH₂;
c) dehydroabietic acid derivatives according to formula I and all stereoisomers thereof, wherein R₁₂ is —Cl, R₁₁ and R₁₄ is —H, R₃ is isopropyl, and R₇ is selected from —H, ═O and ═N—O—R₁, where R₁ is selected from —H, —CH₃, and —CH₂—CH═CH₂;
d) dehydroabietic acid derivatives according to formula I and all stereoisomers thereof, wherein R₁₁ and R₁₂ is —H, R₃ is isopropyl, R₁₄ is —Cl, and R₇ is selected from —H, ═O and ═N—O—R₁, where R₁ is selected from —H, —CH₃, and —CH₂—CH═CH₂;

e) dehydroabietic acid derivatives according to formula I and all stereoisomers thereof, wherein R₁₁ is —Cl, R₃ is isopropyl, and R₇ and R₇ is selected from —H, ═O and ═N—O—R₁, where R₁ is selected from —H, —CH₃, and —CH₂—CH═CH₂, R₁₂ and R₁₄ are —H;
f) dehydroabietic acid derivatives according to formula I and all stereoisomers thereof, wherein R₁₁ and R₁₄ are ═Cl, R₁₂ is —H, R₃ is isopropyl and R₇ is selected from —H, ═O and ═N—O—R₁, where R₁ is selected from —H, —CH₃, and —CH₂—CH═CH₂;
g) dehydroabietic acid derivatives, according to formula I and all stereoisomers thereof wherein R₁₁ and R₁₄ is —H, R₁₂ is —Br, R₃ is isopropyl, and R₇ is selected from —H, ═O, ═N—O—R₁, where R₁ is selected from —H, —CH₃, and —CH₂—CH═CH₂;
h) dehydroabietic acid derivatives according to formula I and all stereoisomers thereof, wherein R₁₁ and R₁₂ is —H, R₃ is isopropyl, R₁₄ is —Br, and R₇ is selected from —H, ═O and ═N—O—R₁, where R₁ is selected from —H, —CH₃, and —CH₂—CH═CH₂;
i) dehydroabietic acid derivatives according to formula I and all stereoisomers thereof, wherein R₁₂ is —I, R₁₁ and R₁₄ is —H, R₃ is isopropyl, and R₇ is selected from —H, ═O and ═N—O—R₁, where R₁ is selected from —H, —CH₃, and —CH₂—CH═CH₂;
j) dehydroabietic acid derivatives according to formula I and all stereoisomers thereof, wherein R₁₁ and R₁₂ is —H, R₁₄ is —I, R₃ is isopropyl, and R₇ is selected from —H, ═O and ═N—O—R₁, where R₁ is selected from —H, —CH₃, and —CH₂—CH═CH₂;
k) dehydroabietic acid derivatives according to formula I and all stereoisomers thereof, wherein R₁₁ is —H, R₁₂ and R₁₄ is —Cl, R₃ is isopropyl, and R₇ is selected from —H, ═O and ═N—O—R₁, where R₁ is selected from —H, —CH₃, and —CH₂—CH═CH₂;
l) dehydroabietic acid derivatives according to formula I and all stereoisomers thereof, wherein R₁₁, R₁₂ and R₁₄ is —Cl, R₃ is isopropyl, and R₇ is selected from —H, ═O, and ═N—O—R₁, where R₁ is selected from —H, —CH₃, and —CH₂—CH═CH₂; and
m) dehydroabietic acid derivative according to formula I and all stereoisomers thereof wherein R₁₁, R₁₂ and R₁₄ is —H, R₃ is isopropyl, and R₇ is selected from —H, ═O, —OH, and ═N—O—R₁, where R₁ is selected from —H, —CH₃, and —CH₂—CH═CH₂.

In one aspect the dehydroabietic acid derivatives are used as defined above wherein R₁₁, R₁₂, and R₁₄ are independently selected from hydrogen and halogen, R₃ is isopropyl; and R₇ is —H, ═N—O—CH₃ or ═N—O—CH₂—CH═CH₂ with the proviso that R₁₂ is not Br.

In one aspect the dehydroabietic acid derivatives for use as defined above are selected from the group of:
(1R,4aS,E)-9-((allyloxy)imino)-6-chloro-7-isopropyl-1,4a-dimethyl-1,2,3,4,4a,9,10,10a-octahydrophenanthrene-1-carboxylic acid;
1R,4aS,E)-6-fluoro-7-isopropyl-9-(methoxyimino)-1,4a-dimethyl-1,2,3,4,4a,9,10,10a-octahydrophenanthrene-1-carboxylic acid;
(1R,4aS,E)-6-chloro-7-isopropyl-9-methoxyimino-1,4a-dimethyl-1,2,3,4,4a,9,10,10a-octahydrophenanthrene-1-carboxylic acid;
(1R,4aS,10aR)-6,8-dichloro-7-isopropyl-9-(methoxyimino)-1,4a-dimethyl-1,2,3,4,4a,9,10,10a-octahydrophenanthrene-1-carboxylic acid;
(1R,4aS,E)-9-((allyloxy)imino)-6-fluoro-7-isopropyl-1,4a-dimethyl-1,2,3,4,4a,9,10,10a-octahydrophenanthrene-1-carboxylic acid;
(1R,4aS,E)-9-((allyloxy)imino)-8-chloro-7-isopropyl-1,4a-dimethyl-1,2,3,4,4a,9,10,10a-octahydrophenanthrene-1-carboxylic acid;
(1R,4aS,E)-8-chloro-7-isopropyl-9-methoxyimino-1,4a-dimethyl-1,2,3,4,4a,9,10,10a-octahydrophenanthrene-1-carboxylic acid;
(1R,4aS)-8-chloro-7-isopropyl-1,4a-dimethyl-1,2,3,4,4a,9,10,10a-octahydrophenanthrene-1-carboxylic acid;
(1R,4aS)-6-chloro-7-isopropyl-1,4a-dimethyl-1,2,3,4,4a,9,10,10a-octahydrophenanthrene-1-carboxylic acid;
(1R,4aS)-8-bromo-7-isopropyl-1,4a-dimethyl-1,2,3,4,4a,9,10,10a-octahydrophenanthrene-1-carboxylic acid;
(1R,4aS,E)-6-iodo-7-isopropyl-1,4a-dimethyl-1,2,3,4,4a,9,10,10a-octahydrophenanthrene-1-carboxylic acid;
1R,4aS)-5-chloro-7-isopropyl-9-(methoxyimino)-1,4a-dimethyl-1,2,3,4,4a,9,10,10a-octahydrophenanthrene-1-carboxylic acid;
(1R,4aS,E)-9-((allyloxy)imino)-6-iodo-7-isopropyl-1,4a-dimethyl-1,2,3,4,4a,9,10,10a-octahydrophenanthrene-1-carboxylic acid;
(1R,4aS,E)-6-iodo-7-isopropyl-9-(methoxyimino)-1,4a-dimethyl-1,2,3,4,4a,9,10,10a-octahydrophenanthrene-1-carboxylic acid;
(1R,4aS,E)-9-(hydroxyimino)-6-iodo-7-isopropyl-1,4a-dimethyl-1,2,3,4,4a,9,10,10a-octahydrophenanthrene-1-carboxylic acid;
(1R,4aS,10aR)+8-iodo-7-isopropyl-9-methoxyimino-1,4a-dimethyl-1,2,3,4,4a,9,10,10a-octahydrophenanthrene-1-carboxylic acid; and
(1R,4aS,E)-8-iodo-7-isopropyl-1,4a-dimethyl-1,2,3,4,4a,9,10,10aoctahydrophenanthrene-1-carboxylic acid.

According to one aspect of the invention the dehydroabietic acid derivatives are used as defined above, wherein R₇ is —H, ═N—O—CHs or ═N—O—CH₂—CH═CH₂; R₃ is isopropyl; and R₁₁, R₁₂ and R₁₄ independently are selected from —H, —F, —Cl, —Br.

According to one aspect of the invention the dehydroabietic acid derivatives are used as defined above and selected from the group of:
(1R,4aS,E)-6-bromo-7-isopropyl-9-(methoxyimino)-1,4a-dimethyl-1,2,3,4,4a,9,10,10a-octahydrophenanthrene-1-carboxylic acid; and
(1R,4aS,E)-6-bromo-9-(hydroxyimino)-7-isopropyl-1,4a-dimethyl-1,2,3,4,4a,9,10,10a-octahydrophenanthrene-1-carboxylic acid.

According to one aspect of the invention the dehydroabietic acid derivatives are used as defined above, wherein R₇ is ═O, ═N—O—CHs or ═N—O—CH₂—CH═CH₂; R₃ is isopropyl; and R₁₁, R₁₂ and R₁₄ independently are selected from—hydrogen and halogen with the proviso that R₁₂ is not bromo. In one embodiment of this aspect the halogen is iodo, preferably R₁₂ is iodo.

According to one aspect of the invention the dehydroabietic acid derivatives are used as defined above and selected from the group of:
(1R,4aS,E)-6-iodo-7-isopropyl-9-oxo-1,4a-dimethyl-1,2,3,4,4a,9,10,10a-octahydrophenanthrene-1-carboxylic acid;
(1R,4aS,E)-6-iodo-7-isopropyl-9-(methoxyimino)-1,4a-dimethyl-1,2,3,4,4a,9,10,10a-octahydrophenanthrene-1-carboxylic acid;
(1R,4aS,E)-8-iodo-7-isopropyl-9-oxo-1,4a-dimethyl-1,2,3,4,4a,9,10,10a-octahydrophenanthrene-1-carboxylic acid; and
(1R,4aS,E)-9-(hydroxyimino)-6-iodo-7-isopropyl-1,4a-dimethyl-1,2,3,4,4a,9,10,10a-octahydrophenanthrene-1-carboxylic acid.

According to one aspect of the invention the dehydroabietic acid derivatives are used as defined above, wherein R₇ is selected from hydrogen, halogen, and ═N—O—R₁ and where R₁₃ is selected from H or halogen. In one embodiment of this aspect the halogen is chloro.

According to one aspect of the invention the dehydroabietic acid derivatives are used as defined above and selected from the group of:
(1R,4aS)-6,7,8-trichloro-1,4a-dimethyl-1,2,3,4,4a,9,10,10a-octahydrophenanthrene-1-carboxylic acid,
(1R,4aS)-7,8-dichloro-1,4a-dimethyl-1,2,3,4,4a,9,10,10a-octahydrophenanthrene-1-carboxylic acid, and
(1R,4aS)-5,6,7,8-tetrachloro-1,4a-dimethyl-1,2,3,4,4a,9,10,10a-octahydrophenanthrene-1-carboxylic acid.

According to one aspect of the invention the dehydroabietic acid derivatives are used as defined above and selected from the group of:
(1S,4aS)-6-chloro-1,4a-dimethyl-1,2,3,4,4a,9,10,10a-octahydrophenanthrene-1-carboxylic acid, and
(1S,4aS)-7-chloro-1,4a-dimethyl-1,2,3,4,4a,9,10,10a-octahydrophenanthrene-1-carboxylic acid.

According to one aspect of the invention the dehydroabietic acid derivatives are used as defined above, wherein R₁₁ and R₁₄ are hydrogen R₁₂ is R₂, R₁₃ is R₃, and R₇═N—O—R₁.

According to one aspect of the invention the dehydroabietic acid derivatives are used as defined above and selected from the group of:
(1R,4aS,E)-9-(methoxyimino)-7-isopropyl-1,4a-dimethyl-6-vinyl-1,2,3,4,4a,9,10,10a-octahydrophenanthrene-1-carboxylic acid,
(1R,4aS,E)-9-((allyloxy)imino)-7-isopropyl-1,4a-dimethyl-6-vinyl-1,2,3,4,4a,9,10,10a-octahydrophenanthrene-1-carboxylic acid,
(1R,4aS,E)-6-cyclopropyl-7-isopropyl-9-(methoxyimino)-1,4a-dimethyl-1,2,3,4,4a,9,10,10a-octahydrophenanthrene-1-carboxylic acid, and
(1R,4aS,E)-9-((allyloxy)imino)-6-cyclopropyl-7-isopropyl-1,4a-dimethyl-1,2,3,4,4a,9,10,10a-octahydrophenanthrene-1-carboxylic acid.

According to one aspect of the invention the dehydroabietic acid derivatives are used as defined above, wherein R₁₁ is hydrogen, R₁₂ and R₁₄ is selected from hydrogen and halogen R₃ is isopropyl, and R₇ is carbonyl.

According to one aspect of the invention the dehydroabietic acid derivatives are used as defined above, wherein R₁₁ is hydrogen, R₁₂ and R₁₄ is selected from hydrogen and —I, R₃ is isopropyl, and R₇ is carbonyl or ═N—OH.

According to yet another aspect, the present invention relates to all compounds presented in table 1 for use in treatment of cardiac arrhythmia or a hyperexcitability disease as defined above by extracellularly acting on the voltage-sensor domain (VSD) to open at least one member of the family of voltage-gated (potassium) channels (Kv family).

According to yet another aspect, the present invention relates to all compounds as earlier defined, for use in the manufacturing of medicament for the treatment of cardiac arrhythmia or a hyperexcitability disease as defined above by extracellularly acting on the voltage sensitive domain (VSD) to open at least one member of the family of voltage-gated (potassium) channels (Kv family).

According to still yet another aspect, the present invention relates to a method of treating cardiac arrhythmia or a hyperexcitability disease as defined above by extracellularly acting on the voltage sensitive domain (VSD) to open at least one member of the family of voltage-gated (potassium) channels (Kv family), by administering at least one dehydroabietic acid derivative as defined above in therapeutically effective amount.

According to a different aspect the invention relates to dehydroabietic acid derivatives according to formula I and all stereoisomers thereof,

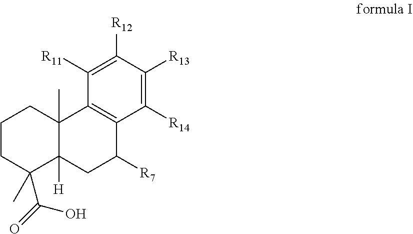

selected from the compounds included in the groups a) to m):
a) dehydroabietic acid derivatives according to formula I and all stereoisomers thereof, wherein R₁₂ is —F, R₁₁ and R₁₄ is —H, R₁₃ is isopropyl, and R₇ is selected from —H, ═O and ═N—O—R₁, where R₁ is selected from —H, —CH₃, —CH₂—CH═CH₂, and —CH₂—C₆H₅;
b) dehydroabietic acid derivatives according to formula I and all stereoisomers thereof, wherein R₁₁ and R₁₂ is —H, R₁₄ is —F, R₁₃ is isopropyl, and R₇ is selected from —H, ═O and ═N—O—R₁, where R₁ is selected from —CH₃, —CH₂—CH═CH₂, and CH₂—C₆H₅;
c) dehydroabietic acid derivatives according to formula I and all stereoisomers thereof, wherein R₁₂ is ═Cl, R₁₁ and R₁₄ is —H, R₁₃ is isopropyl, and R₇ is selected from —H, ═O and ═N—O—R₁, where R₁ is selected from —H, —CH₃, —CH₂—CH═CH₂, and —CH₂—C₆H₅;
d) dehydroabietic acid derivatives according to formula I and all stereoisomers thereof, wherein R₁₁ and R₁₂ is —H, R₁₃ is isopropyl, R₁₄ is —Cl, and R₇ is selected from —H, ═O and ═N—O—R₁, where R₁ is selected from —H, —CH₃, —CH₂—CH═CH₂, and —CH₂—C₆H₅;
e) dehydroabietic acid derivatives according to formula I and all stereoisomers thereof, wherein R₁₁ is ═Cl, R₁₃ is isopropyl, R₇ is selected from —H, ═O and ═N—O—R₁, where R₁ is selected from —H, —CH₃, —CH₂—CH═CH₂, and —CH₂—C₆H₅; and R₁₂ and R₁₄ are —H;

f) dehydroabietic acid derivatives according to formula I and all stereoisomers thereof, wherein R₁₁ and R₁₄ is —H, R₁₂ is —Br, R₁₃ is isopropyl, and R₇ is selected from ═O and ═N—O—CH₂—C₆H₅;
g) dehydroabietic acid derivatives according to formula I and all stereoisomers thereof, wherein R₁₁ and R₁₂ is —H, R₁₃ is isopropyl, R₁₄ is —Br, and R₇ is selected from ═O and ═N—O—R₁, where R₁ is selected from —H, —CH₂—CH═CH₂, and —CH₂—C₆H₅;
h) dehydroabietic acid derivatives according to formula I and all stereoisomers thereof, wherein R₁₂ is —I, R₁₁ and R₁₄ is —H, R₁₃ is isopropyl, and R₇ is selected from —H, ═O and ═N—O—R₁, where R₁ is selected from —H, —CH₃, —CH₂—CH═CH₂, and —CH₂—C₆H₅;
i) dehydroabietic acid derivatives according to formula I and all stereoisomers thereof, wherein R₁₁ and R₁₂ is —H, R₁₃ is isopropyl, R₁₄ is —I, and R₇ is selected from ═O and ═N—O—R₁, where R₁ is selected from —H, —CH₃, —CH₂—CH═CH₂, and —CH₂—C₆H₅;

j) dehydroabietic acid derivatives according to formula I and all stereoisomers thereof, wherein R₁₁, R₁₂ and R₁₄ is —Cl, R₁₃ is isopropyl, and R₇ is selected from —H, ═O, and ═N—O—CH₂—C₆H₅;
k) dehydroabietic acid derivatives according to formula I and all stereoisomers thereof, wherein R₁₁ and R₁₂ are independently selected from hydrogen and halogen, R₇ is selected from hydrogen, halogen and ═N—O—R₁, wherein R₁ is selected from —H, —CH₃, —CH₂—CH═CH₂, and —CH₂—C₆H₅ and wherein R₁₃ is selected from H or halogen;
l) dehydroabietic acid derivatives according to formula I and all stereoisomers thereof, wherein R₁₁ and R₁₄ are hydrogen R₁₂ is a straight, branched or cyclic saturated or unsaturated hydrocarbon comprising from 1 to 6 carbon atoms (C1-C6 alkyl, C2-C6 alkenyl and C3-C6 cycloalkyl; said alkyl, alkenyl, and cycloalkyl optionally being substituted with at least one halogen), R₁₃ is isopropyl, and R₇═N—O—R₁, R₁ is selected from —H, —CH₃, and —CH₂—CH═CH₂, and —CH₂—C₆H₅; and
m) dehydroabietic acid according to formula I and all stereoisomers thereof, wherein R₁₁ is hydrogen, R₁₂ and R₁₄ is selected from hydrogen and iodo, R₃ is isopropyl, and R₇ is ═O.

In this aspect “dehydroabietic acid derivatives according to formula I and all stereoisomers thereof” is given the same meaning as above and would include also derivatives of podocarpic acid.

According to one aspect, the dehydroabietic acid derivatives are defined as above, wherein R₇ is selected from ═N—O—R₁, and R₁ is selected from —CH₃ and —CH₂—CH═CH₂.

According to one aspect, the dehydroabietic acid derivatives from are included in groups a) to m) above and selected from:
(1R,4aS)-8-chloro-7-isopropyl-1,4a-dimethyl-1,2,3,4,4a,9,10,10a-octahydrophenanthrene-1-carboxylic acid;
(1R,4aS)-6-chloro-7-isopropyl-1,4a-dimethyl-1,2,3,4,4a,9,10,10a-octahydrophenanthrene-1-carboxylic acid;
(1R,4aS,E)-8-chloro-7-isopropyl-9-methoxyimino-1,4a-dimethyl-1,2,3,4,4a,9,10,10a-octahydrophenanthrene-1-carboxylic acid;
(1R,4aS,E)-9-((allyloxy)imino)-8-chloro-7-isopropyl-1,4a-dimethyl-1,2,3,4,4a,9,10,10a-octahydrophenanthrene-1-carboxylic acid;
(1R,4aS,E)-6-chloro-7-isopropyl-9-oxo-1,4a-dimethyl-1,2,3,4,4a,9,10,10a-octahydrophenanthrene-1-carboxylic acid;
(1R,4aS,E)-6-chloro-7-isopropyl-9-hydroxyimino-1,4a-dimethyl-1,2,3,4,4a,9,10,10a-octahydrophenanthrene-1-carboxylic acid;
(1R,4aS,E)-9-((allyloxy)imino)-6-chloro-7-isopropyl-1,4a-dimethyl-1,2,3,4,4a,9,10,10a-octahydrophenanthrene-1-carboxylic acid;
(1R,4aS,E)-6-bromo-7-isopropyl-1,4a-dimethyl-1,2,3,4,4a,9,10,10a-octahydrophenanthrene-1-carboxylic acid;
(1R,4aS)-8-bromo-7-isopropyl-1,4a-dimethyl-1,2,3,4,4a,9,10,10a-octahydrophenanthrene-1-carboxylic acid;
(1R,4aS,E)-9-((allyloxy)imino)-6-fluoro-7-isopropyl-1,4a-dimethyl-1,2,3,4,4a,9,10,10a-octahydrophenanthrene-1-carboxylic acid;
(1R,4aS,E)-6-fluoro-7-isopropyl-9-(methoxyimino)-1,4a-dimethyl-1,2,3,4,4a,9,10,10a-octahydrophenanthrene-1-carboxylic acid;
(1R,4aS,E)-6-iodo-7-isopropyl-1,4a-dimethyl-1,2,3,4,4a,9,10,10a-octahydrophenanthrene-1-carboxylic acid;
(1R,4aS,E)-9-((allyloxy)imino)-6-iodo-7-isopropyl-1,4a-dimethyl-1,2,3,4,4a,9,10,10a-octahydrophenanthrene-1-carboxylic acid;
(1R,4aS,E)-6-iodo-7-isopropyl-9-(methoxyimino)-1,4a-dimethyl-1,2,3,4,4a,9,10,10a-octahydrophenanthrene-1-carboxylic acid;
(1R,4aS,E)-6-chloro-7-isopropyl-9-methoxyimino-1,4a-dimethyl-1,2,3,4,4a,9,10,10a-octahydrophenanthrene-1-carboxylic acid;
(1R,4aS)-5-chloro-7-isopropyl-9-(methoxyimino)-1,4a-dimethyl-1,2,3,4,4a,9,10,10a-octahydrophenanthrene-1-carboxylic acid;
zo (1R,4aS)-6,7,8-trichloro-1,4a-dimethyl-1,2,3,4,4a,9,10,10a-octahydrophenanthrene-1-carboxylic acid;
(1R,4aS)-7,8-dichloro-1,4a-dimethyl-1,2,3,4,4a,9,10,10a-octahydrophenanthrene-1-carboxylic acid;
(1R,4aS)-5,6,7,8-tetrachloro-1,4a-dimethyl-1,2,3,4,4a,9,10,10a-octahydrophenanthrene-1-carboxylic acid;
(1R,4aS,E)-9-(methoxyimino)-7-isopropyl-1,4a-dimethyl-6-vinyl-1,2,3,4,4a,9,10,10a-octahydrophenanthrene-1-carboxylic acid;
(1R,4aS,E)-9-((allyloxy)imino)-7-isopropyl-1,4a-dimethyl-6-vinyl-1,2,3,4,4a,9,10,10a-octahydrophenanthrene-1-carboxylic acid;
(1R,4aS,E)-6-cyclopropyl-7-isopropyl-9-(methoxyimino)-1,4a-dimethyl-1,2,3,4,4a,9,10,10a-octahydrophenanthrene-1-carboxylic acid;
(1R,4aS,E)-9-((allyloxy)imino)-6-cyclopropyl-7-isopropyl-1,4a-dimethyl-1,2,3,4,4a,9,10,10a-octahydrophenanthrene-1-carboxylic acid;
(1R,4aS,E)-8-iodo-7-isopropyl-1,4a-dimethyl-1,2,3,4,4a,9,10,10a-octahydrophenanthrene-1-carboxylic acid;
(1R,4aS,E)-9-(hydroxyimino)-6-iodo-7-isopropyl-1,4a-dimethyl-1,2,3,4,4a,9,10,10a-octahydrophenanthrene-1-carboxylic acid;
(1R,4aS,10aR)−)-8-iodo-7-isopropyl-9-methoxyimino-1,4a-dimethyl-1,2,3,4,4a,9,10,10a-octahydrophenanthrene-1-carboxylic acid;
(1S,4aS)-1,4a-dimethyl-1,2,3,4,4a,9,10,10a-octahydrophenanthrene-1-carboxylic acid
(1S,4aS)-6-chloro-1,4a-dimethyl-1,2,3,4,4a,9,10,10a-octahydrophenanthrene-1-carboxylic acid; and
(1S,4aS)-7-chloro-1,4a-dimethyl-1,2,3,4,4a,9,10,10a-octahydrophenanthrene-1-carboxylic acid.

Table 1 below show compounds according to the invention that can be useful for treatment of cardiac arrhythmia, or a hyperexcitability disease, such as pain or epilepsy when administered in a therapeutically acceptable dose.

### BRIEF DESCRIPTION OF THE DRAWINGS

FIGS. 1A-F show the effect of several natural resin acids on the opening of the Shaker K channel.

FIGS. 2A-C show the efficacy of DHAA according to the invention modified at C7 at the B-ring to activate 3R Shaker K channel in comparison to DHAA.

FIGS. 3A-C show potency variations for halogen modification of DHAA-derivatives according to the invention.

FIGS. 4A-C show a comparison between DHAA derivatives according to the invention modified in C13.

FIGS. 5A-D show dose and pH dependent compound sensitivity comparisons for DHAA and DHAA derivatives according to the invention.

FIG. 6 shows correlations between the G(V) shifts for the 3R channel expressed in CHO-cells (10 μM at pH 7.4) versus Xenopus oocytes (100 μM at pH 7.4).

FIGS. 7A-D shows the effect of DHAA derivatives on the resting potential and excitability of DRG neurons.

FIG. 8 shows the membrane potential of a spontaneously beating HL-1 cell. 10 uM of the (1R,4aS,E)-6-chloro-7-isopropyl-9-methoxyimino-1,4a-dimethyl-1,2,3,4,4a,9,10,10a-octahydrophenanthrene-1-carboxylic acid (Wu69) hyperpolarized the membrane potential and reduced the frequency.

### DETAILED AND EXPERIMENTAL DESCRIPTION OF THE INVENTION

In the context of the present invention, both when it is described in the previous generalized aspects and in the detailed forms in the following experimental part, the following definitions can be used:

An ion channel is broadly defined as a transmembrane molecule allowing ions to passively and (in most cases) selectively pass across the cellular membrane. Ion channels are classified according to their genetic homology.

A voltage-gated ion channel or a voltage-gated channel is herein defined as a distinct class of ion channels that sense the cellular membrane voltage (Vₘ) to let it control the gate that opens and closes the ion-conducting pore. The members of the superfamily of voltage-gated ion channels are found at http://www.guidetopharmacology.org/GRAC/FamilyDisplayForward?familyId=696&familyType=IC#subfamilies. The superfamily has seven families: (1) CatSper and Two-Pore channels, (2) Cyclic nucleotide-regulated channels, (3) K (=potassium) channels (a, Calcium-activated K channels, b, Inwardly rectifying K channels, c, Two-P K channels, d, Voltage-gated K channels), (4) Transient Receptor Potential channels, (5) Voltage-gated calcium channels, (6) Voltage-gated proton channel, (7) Voltage-gated sodium channels. The channels' building blocks have a core structure consisting of six transmembrane (6TM) segments named segment 1 (S1) to segment 6 (S6).

A voltage-gated K ion channel (hereinafter called Kv channel) belongs to the family of Kv channel (see 3d above). The family of Kv channels has 12 subfamilies, Kv1 to Kv12. Among all 143 human voltage-gated ion channels, the voltage-gated K (Kv) channels belonging to the subfamilies Kv1-Kv9 form a branch on its own in the ion channel tree, suggesting a close functional kinship.

Kv1, Kv2, Kv3, Kv 4, and Kv7 represent subfamilies of the nine subfamilies that form functional ion channels on their own. These types of ion channels exist in neurons; mutations in some of them cause epilepsy, and opening of the ion conducting pore could potentially reduce excitability and treat epilepsy or pain.

A Shaker K channel is a Kv channel from the fruit fly Drosophila melanogaster. It is homologous to the human Kv1-type ion channels.

A 3R Shaker K channel is a modified Shaker K channel where residues 356 and 359 are mutated to arginines.

A voltage-sensor domain (VSD) of a voltage-gated ion channel is composed of S1 to S4. The voltage sensor S4 has three to eight positively charged amino acid residues, with two hydrophobic residues between each positively charged residue.

Opening of a Kv channel is caused by the movement of S4. At a normal resting potential (Vₘ≈−70 mV) S4 is in a down state, that is, the charged amino-acid residues are close to the intracellular side of the Kv channel. The channel is in a resting state. When the membrane potential becomes more positive, S4 moves to an upstate, that is, the charged amino-acid residues are close to the extracellular side of the Kv channel. This state is called the activated state. From the activated state the gate that opens and closes the channel is spontaneously pulled open and the channel conducts ions. This is called the open state.

Extracellular activator of voltage-gated K channels refers to a compound acting via the extracellular part of the ion channel. Such compounds can either bind to the lipid bilayer and from this position activate the channel or it can bind directly to the ion channel's extracellular portion, thereby opening the channel.

Affecting or shifting the voltage dependence of voltage-gated Kv channel has the meaning of shifting the conductance-versus-voltage, G(V), curve along the voltage axis. K conductance (G_{K}) is calculated as G_{K}=I_{K}/(Vₘ−V_{K}), where I_{K} is the K current and V_{K} is the equilibrium potential for the K ion, close to −90 mV. A shift in negative direction along the voltage axis results in an opening of the ion channel at a specific voltage.

Potency or “a potent shifter” when referred to tested compounds in the present context has the meaning of a G(V) shift larger (in absolute terms) than −20 mV at 100 μM and pH 7.4.

Hyperpolarization is defined as a deviation of the membrane potential in the negative direction.

Excitability has the meaning of regenerative electrical activity (a so called action potential) of a cell or cell membrane. That is, a highly non-linear membrane voltage response in relation to the current injected to the cell.

Hyperexcitability is a condition of increased excitability, higher than in a normal cell. A smaller current is needed to be injected into the cell to cause an action potential. There are a number of medical conditions with increased excitability, for instance epilepsy, cardiac arrhythmia, multiple sclerosis and pain.

Hypoexcitability is a condition of reduced excitability. A number of muscle diseases like hyperkalemic periodic paralysis and paramyotonia congenita belong to these conditions.

### Methods

### Shaker K Channels

All animal experiments were approved by the local Animal Care and Use Committee and followed international guidelines. A modified Shaker H4 channel³⁰, with removed N-type inactivation (ShH4IR)³¹, here called the wild type (WT) channel, was expressed in oocytes. In addition, a modified 3R Shaker K channel where two introduced positive charged arginines (M356R and A359R) in addition to one native arginine (R362) makes the channel more sensitive to DHA¹⁶, was expressed in oocytes and in a CHO—K1 stable cell line.

### Preparation and Injection of Oocytes

African clawed frogs (Xenopus laevis) were anesthetized with 1.4 g/L ethyl 3-aminobenzoate methanesulfonate salt (tricaine). After an incision through the abdomen a batch of oocytes were removed. Clusters of oocytes were separated by incubation for ˜1 h in a Ca-free O—R₂ solution (in mM: 82.5 NaCl, 2 KCl, 5 HEPES, and 1 MgCl₂; pH adjusted to 7.4 by NaOH) containing Liberase Blendzyme. The oocytes were then incubated at 8° C. in a modified Barth's solution (MBS; in mM: 88 NaCl, 1 KCl, 2.4 NaHCO₃, 15 HEPES, 0.33 Ca(NO₃)₂, 0.41 CaCl₂, and 0.82 MgSO₄; pH adjusted to 7.6 by NaOH) supplemented with penicillin (25 U/ml), streptomycin (25 μg/ml), and sodium pyruvate (2.5 mM) 2-24 hours before injection. Fifty nl of cRNA (50 pg) were injected into each oocyte using a Nanoject injector (Drummond Scientific, Broomall, Pa.). Injected oocytes were kept at 8° C. in MBS until one day before electrophysiological recordings, when they were incubated at 16° C. All chemicals were supplied from Sigma-Aldrich (Stockholm, Sweden) if not stated otherwise.

### Generation and Maintenance of CHO—K1 Cell Lines Stably Expressing 3R Channels

For the modified 3R Shaker K channel a stable cell line was generated. The construct was cloned into the pcDNA3 vector. CHO—K1 cells were plated into T75 culture flasks and grown without penicillin/streptomycin. After 24 h cells were transfected with 25 μg expression construct and 60 μl Lipofectamin 2000 according to the manufacturer's protocol. For polyclonal selection cells were grown in the presence of G418 (400 μg/ml). Single cells from G418 resistant cell pools were plated into 96 well plates for dilution cloning. Monoclonal cell lines from single colonies were expanded and tested electrophysiologically for ion channel expression.

One stable monoclonal CHO—K1 cell line expressing the 3R Shaker K channel was selected and used for all compound testing. For manual electrophysiology, cells were cultured in F-12 Nutrient Mixture (Ham) with GlutaMAX™ supplemented with 10% fetal calf serum, penicillin/streptomycin and G418 (200 μg/ml) at 37° C. with 5% CO₂. For automated electrophysiology, cells were cultured in DMEM/F12+ Glutamax (Gibco), supplemented with 10% fetal calf serum, 1% non-essential amino acids (Invitrogen), and G418 (400 μg/ml) at 37° C. with 5% CO₂.

Preparation of Dissociated Neurons from Dorsal Root Ganglia

All animal experiments were approved by the local Animal Care and Use Committee and followed international guidelines. Five female 7-12 week-old C57BL/6 mice (Scanbur) were used for this study. The mice were anaesthetized with isoflurane and decapitated. Dorsal root ganglia (DRG) were dissected from all spinal levels and enzymatically digested with collagenase (250 CDU/ml) for 15 min, and then trypsin (1 mg/ml) was added for another 30 min. The ganglia were centrifuged and resuspended in Leibovitz L-15 media with glutamine, supplemented with 10% fetal calf serum, 38 mM glucose, 24 mM NaHCO₃ and penicillin/streptomycin. The ganglia were triturated and the DRG neurons plated on poly-D-lysine coated plastic coverslips. The neurons were cultured at 37° C. with 5% CO₂ for 2-3 days before the electrophysiological recordings.

### Manual Electrophysiology

All manual electrophysiological recordings were performed at room temperature (20-23° C.), using a GeneClamp 500B amplifier (for oocytes) or a Axopatch 200B amplifier (for manual patch clamp of CHO—K1 cells and DRG neurons) and a Digidata 1440A digitizer and pClamp 10 software (all from Molecular Devices, Inc., Sunnyvale, Calif., USA). Compounds were dissolved to 100 mM in 99.5% EtOH and stored at −20° C. Compounds were diluted in extracellular solution to the desired test concentration.

### Manual Two-Electrode Voltage-Clamp (TEVC) of Oocytes

The oocyte was placed in a bath surrounded by 1K extracellular solution that contained (in mM): 88 NaCl, 1 KCl, 15 HEPES, 0.4 CaCl₂, and 0.8 MgCl₂, pH adjusted to 7.4 by NaOH (reaching a sodium concentration of ˜100 mM). Control solution was added to the bath with a gravity driven perfusion system. Compound solution was added to the bath manually with a syringe. Two glass microelectrodes were inserted into the oocyte using micromanipulators. The microelectrodes were pulled from borosilicate glass, filled with 3M KCl and had a resistance of 0.5-2 MO. All channels were closed when the potential was clamped to −80 mV and this voltage was set as the holding potential. Currents were evoked from the holding potential of −80 mV by 100-ms long, 5-mV steps ranging from −80 up to +50 mV (WT) and +70 mV (3R).

### Manual Whole-Cell Patch Clamp of CHO—K1 Cells and DRG Neurons

CHO—K1 Cells were plated on coverslips 2 h before the manual electrophysiological recordings. Coverslips with CHO—K1 cells or DRG neurons were placed in a recording chamber and perfused with extracellular solution by a gravity-fed perfusion system. Substances were applied by a pressurized, automated OctaFlow perfusion system (ALA Scientific Instruments). The signals were sampled at 5-20 kHz after low-pass filtering at 2-5 kHz. For CHO—K1 cells the intracellular solution contained (in mM): 120 K-gluconate, 10 KCl, 5 EGTA, 10 HEPES, 4 Mg-ATP, 0.3 Na-GTP, pH 7.3; the extracellular solution contained (in mM): 135 NaCl, 4 KCl, 10 HEPES, 1 MgCl₂, 1.8 CaCl₂, 10 glucose, pH 7.4. Currents were evoked in CHO—K1 cells in whole-cell voltage-clamp mode from a holding potential of −80 mV by 100-ms long, 10-mV steps ranging from −80 to +80 mV.

For DRG neurons the intracellular solution contained (in mM): 120 K-gluconate, 10 KCl, 1 EGTA, 10 HEPES, 4 Mg-ATP, 0.3 Na-GTP, pH 7.3; the extracellular solution contained (in mM): 144 NaCl, 2.5 KCl, 10 HEPES, 0.5 MgCl₂, 2 CaCl₂, 10 glucose, pH 7.4. A liquid-junction potential of approximately −14 mV was corrected for. Pipettes were pulled from borosilicate glass with a vertical patch electrode puller PIP5 (HEKA) and had a resistance of 4-6 MΩ when filled with intracellular solution. Small and medium sized DRG neurons were selected for whole-cell current-clamp recording. 10 μM of the compounds were applied for 120 s to study the effect on the resting membrane potential, and for 45-120 s to study the effects on evoked action potentials. To evoke action potentials 800-ms depolarizing pulses

### Analysis of Electrophysiological Data

The manual electrophysiological data from oocytes, CHO—K1 cells and DRG neurons were processed with Clampfit 10.4 (Molecular Devices, LLC.) and GraphPad Prism 5 (GraphPad Software, inc).

### Analysis of Oocyte Recordings and Manual Whole Cell Recordings of CHO—K1 Cells

Conductance G_{K}(V) was calculated as

G_{K}(V)=I_{K}(V−Vᵣₑᵥ), (Eq. 1)

where I_{K} is the average current from the steady-state phase at the end of each 100-ms pulse, V is the membrane voltage, and Vᵣₑᵥ is the reversal potential for K⁺, (set to −80 mV for the oocytes, and calculated to be −89 mV for the CHO—K1 cells). These data were fitted to a Boltzmann equation

G(V)=A/(1+exp((V_{1/2}−V)/s))ⁿ (Eq. 2)

where A is amplitude of the curve, V is the absolute membrane voltage, V_{1/2} is the midpoint, s is the slope and n is an exponent set to 4¹⁷. The compound-induced G(V) shift was determined at the 10% level of the maximum conductance in control solution¹⁷. The compound-induced G(V) shift in CHO—K1 was quantified by subtracting the control V_{1/2} from the compound V_{1/2} when n was set to 1.

Data for concentration and pH dependent G(V) shifts (ΔV) in oocytes were fit with a dose-response equation

ΔV=ΔVₘₐₓ/(1+c_{1/2}/c), (Eq. 3)

where Δvₘₐₓ is the maximal shift, c_{1/2} is half maximal effective concentration/pKₐ value and c is the concentration.

### Analysis of Manual Whole-Cell Recordings of DRG Neurons

The compound effect on the resting membrane potential Vₘ was determined by subtracting the control pre-compound Vₘ from the Vₘ at the end of the 120 s application of test compound.

### Statistical Analysis

Average values are expressed as mean±SEM. When comparing compound-induced shifts of mutants with control (R362Q) one-way ANOVA together with Dunnett's multiple comparison test was used. When comparing groups, one-way ANOVA together with Bonferroni's multiple comparison tests was used. Correlation analysis was done by Pearson's correlation test and linear regression. P<0.05 is considered significant for all tests.

Synthesis of Compounds of the Invention

### General Methods and Materials

All the solvents and reagents were used without further distillation or drying. The solution of Cl₂ in CCl₄ was prepared at a concentration of 0.26 M. Microwave heated reactions were run in an Initiator instrument from Biotage. Analytical thin-layer chromatography was performed on the Merk silica gel 60F254 glass-backed plates. Flash chromatography was performed with silica gel 60 (particles size (0.040-0.063 mm). Preparative liquid chromatography was run on a Gilson Unipoint system with a Gemini C18 column (100×21.20 mm, 5 micron) under neutral condition using gradient CH3CN/water as eluent (water phase: 95:5 water:acetonitrile, 10 mM NH₄OAc, organic phase 90:10 acetonitrile:water, 10 mM NH₄OAc). NMR spectra were recorded on a Varian Avance 300 MHz and 500 MHz with solvent indicated. Chemical shift was reported in ppm on the δ scale and referenced to the solvent peak. Compounds Wu27, Wu45, K4-K6, K8-K10 were synthesized using the method described in the literature with more than 95% purity. Compounds Wu13, Wu14, Wu24, Wu27, Wu28, Wu30-Wu33, Wu35-Wu37, Wu40-Wu43, Wu45-Wu49, Wu51 were known and synthesized with a similar but optimized method as described in the general procedure. (Cui, Y. M. et al. Design, synthesis, and characterization of BK channel openers based on oximation of abietane diterpene derivatives. Bioorg. Med. Chem., 18 (24), 8642-8659 (2010); Dimitriadis Kutney, J. P. & Dimitriadis, E. Studies related to biological detoxification of kraft pulp mill effluent. V. The synthesis of 12- and 14-chlorodehydroabietic acids and 12,14-dichlorodehydroabietic acid, fish-toxic diterpenes from kraft pulp mill effluent. Helv. Chim. Acta, 65 (5), 1351-1358 (1982).). All known products gave satisfactory analytical and spectroscopic data corresponding to the reported literature values. Because the 12-bromo series compounds Wu32 and Wu33 were important, their analytical data were therefore reported as representatives for other known compounds, which were synthesized with a slightly different but optimal procedure from the literature. Wu50 is a side product from the formation of trichloroDHAA³ (e.g. Wu47). The structure of Wu50 was elucidated from 1D (¹H and ¹³CNMR) and 2D NMR (HSQC, HMBC and COSY) data.

### General Procedure A:

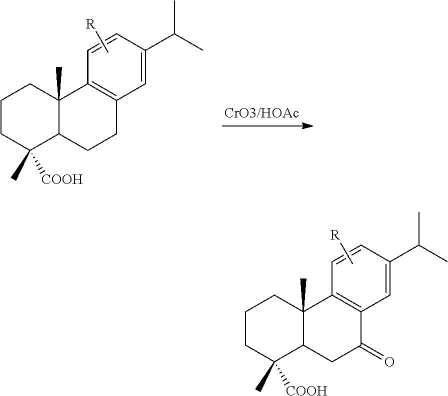

To a solution of halogenated dehydroabietic acid in acetic acid (HOAc) is added a mixture of CrO₃ (1.2-2.0 mole equivalent) in HOAc at room temperature. The mixture is stirred at 50° C. for 3 h to (overnight) 12 h, and concentrated and purified on silica gel or further with preparative HPLC to give a ketone as desired product in 13-59% yield.

### General Procedure B:

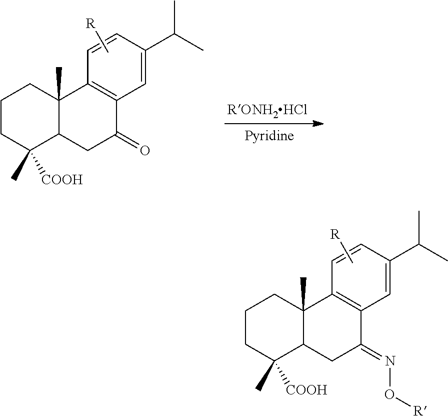

To a mixture of the ketone and 2.0-5.0 mole equivalent R′ONH₂ hydrochloride salt was added 0.7-1.0 mL ethanol (EtOH) followed by 2.1-5.1 mole equivalent pyridine. The mixture was heated under microwave irradiation at 110° C. for 1 h and purified on silica gel with 30-45% ethylacetate-n-heptane (containing 0.1% HCOOH) to give 24-100% yield of oxime as the desired product.

### Synthesis of Compound Wu13 and Wu14

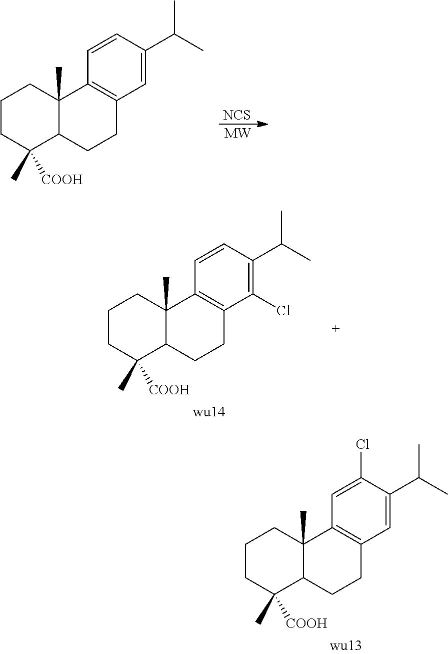

(1R,4aS)-8-chloro-7-isopropyl-1,4a-dimethyl-1,2,3,4,4a,9,10,10a-octahydrophenanthrene-1-carboxylic acid (Wu14) and (1R,4aS)-6-chloro-7-isopropyl-1,4a-dimethyl-1,2,3,4,4a,9,10,10a-octahydrophenanthrene-1-carboxylic acid (Wu13): The mixture of dehydroabietic acid (300.4 mg, 1.0 mMol) and N-chlorosuccinimide (106.8 mg, 0.8 mMol) in 3 mL acetonitrile in a 2-5.0 mL vial was heated under microwave irradiation at 100° C. for 20 min, then another 106.8 mg of N-chlorosuccinimide was added and stirred at 100° C. for another 30 min. Concentrated and purified on silica gel with 20-40% EA-n-heptane (0.1% HCOOH) to give 51.6 mg Wu14 (15% yield) and 139.0 mg Wu13 (yield 42%) as white solid. Wu13: NMR ¹H (300 MHz, CDCl₃) δ 7.19 (s, 1H), 6.94 (s, 1H), 3.32, (m, 1H), 2.95-2.80 (m, 2H), 2.26 (d, J=12.9 Hz, 1H), 2.20 (dd, J=12.3, 2.4 Hz, 1H), 1.90-1.63 (m, 5H), 1.62-1.42 (m, 2H), 1.29 (s, 3H), ?, ?. ¹³C (75 MHz, CDCl₃) δ 185.5, 148.4, 142.6, 133.8, 130.8, 127.2, 125.3, 47.5, 44.4, 37.9, 37.1, 36.8, 29.8, 29.6, 25.1, 22.9, 22.8, 21.7, 18.5, 16.3. Wu14: NMR ¹H (300 MHz, CDCl₃) δ 7.17 (d, J=8.1 Hz, 1H), 7.11 (d, J=8.1 Hz, 1H), 3.49-3.36 (m, 1H), 3.04-2.92 (m, 1H), 2.90-2.71 (m, 1H), 2.30 (br d, J=12.3 Hz, 1H), 2.19 (dd, J=10.5, 2.4 Hz, 1H), 1.92-1.59 (m, 6H), 1.52-1.40 (m, 1H), 1.29 (s, 3H), 1.27-1.16 (m, 9). ¹³C (75 MHz, CDCl₃) δ 184.8, 148.9, 143.0, 133.8, 133.4, 123.7, 122.7, 47.4, 44.0, 38.3, 37.3, 36.7, 30.3, 29.3, 25.2, 23.0, 22.7, 21.6, 18.7, 16.3.

### Synthesis of Wu16

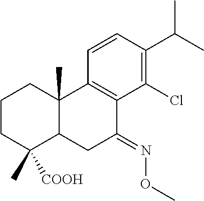 [SMILES: CO/N=C1\CC2[C@](C)(C(=O)O)CCC[C@]2(C)c2ccc(C(C)C)c(Cl)c21]

(1R,4aS,E)-8-chloro-7-isopropyl-9-methoxyimino-1,4a-dimethyl-1,2,3,4,4a,9,10,10a-octahydrophenanthrene-1-carboxylic acid (Wu16): Followed the general procedure B, ketone Wu14 (24.1 mg, 0.069 mMol), O-methylhydroxylamine hydrochloride (11.5 mg, 0.138 mMol) and pyridine (11.5 mg, 0.145 mMol) were used and 12.6 mg product Wu16 (48% yield) was achieved. NMR ¹H (300 MHz, CDCl₃) δ 7.22 (d, J=8.1 Hz, 1H), 7.12 (d, J=8.1 Hz, 1H), 4.04 (s, 3H), 3.54 (m, 1H), 3.01 (dd, J=18.6, 12.6 Hz, 1H), 2.46 (dd, J=18.6, 6.0 Hz, 1H), 2.21 (br d, J=12.3 Hz, 1H), 2.13 (dd, J=12.9, 6.6 Hz, 1H), 1.82-1.58 (m, 5H), 1.39 (s, 3H), 1.25 (d, J=6.6 Hz, 3H), 1.19 (d, J=6.9 Hz, 3H), 1.08 (s, 3H). ¹³C (75 MHz, CDCl₃) δ 182.5, 152.9, 151.4, 145.4, 130.9, 128.5, 126.7, 121.0, 61.4, 46.0, 41.4, 37.8, 37.6, 37.2, 30.4, 24.9, 23.3, 22.6, 21.5, 18.0, 16.8.

### Synthesis of Wu19

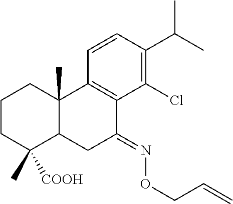 [SMILES: C=CCO/N=C1\CC2[C@](C)(C(=O)O)CCC[C@]2(C)c2ccc(C(C)C)c(Cl)c21]

(1R,4aS,E)-9-((allyloxy)imino)-8-chloro-7-isopropyl-1,4a-dimethyl-1,2,3,4,4a,9,10,10a-octahydrophenanthrene-1-carboxylic acid (Wu19): Followed the general procedure B, ketone Wu14 (21.7 mg, 0.062 mMol), 0-allylhydroxylamine hydrochloride (13.6 mg, 0.124 mMol) and pyridine (9.8 mg, 0.130 mMol) were used and 12.5 mg product Wu19 (50% yield) was achieved. NMR ¹H (300 MHz, CDCl₃) δ 7.22 (d, J=8.7 Hz, 1H), 7.12 (d, J=8.4 Hz, 1H), 6.20-6.02 (m, 1H), 5.35 (d, J=16.5 Hz, 1H), 5.23 (d, J=9.9 Hz, 1H), 4.80-4.65 (m, 2H), 3.53 (m, 1H), 3.05 (dd, J=18.6, 12.9 Hz, 1H), 2.49 (dd, J=18.6, 6.6 Hz, 1H), 2.20 (br d, J=11.1 Hz, 1H), 2.13 (dd, J=12.9, 6.6 Hz, 1H), 1.82-1.58 (m, 5H), 1.38 (s, 3H), 1.25 (d, J=7.2 Hz, 3H), 1.19 (d, J=6.6 Hz, 3H), 1.08 (s, 3H). ¹³C (75 MHz, CDCl₃) δ 183.8, 153.1, 151.3, 145.4, 135.1, 130.9, 128.6, 126.7, 120.9, 117.4, 75.4, 46.1, 41.3, 37.7, 37.6, 37.3, 30.4, 25.0, 23.3, 22.6, 21.5, 18.1, 16.7.

### Synthesis of Wu20

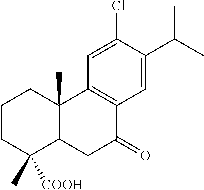 [SMILES: CC(C)c1cc2c(cc1Cl)[C@@]1(C)CCC[C@@](C)(C(=O)O)C1CC2=O]

(1R,4aS,E)-6-chloro-7-isopropyl-9-oxo-1,4a-dimethyl-1,2,3,4,4a,9,10,10a-octahydrophenanthrene-1-carboxylic acid. Followed the general procedure A, 12-chloro dehydroabietic acid (83.0 mg, 0.248 mMol) and CrO₃ (29.7 mg, 0.297 mMol) were used as started material, and 37.0 mg Wu20 was got, yield 43%. NMR ¹H (300 MHz, CDCl₃) δ 7.94 (s, 1H), 7.33 (s, 1H), 3.54 (m, 1H), 2.78-2.61 (m, 2H), 2.49 (d, J=9.0 Hz, 1H), 2.31 (d, J=7.5 Hz, 1H), 1.86-1.73 (m, 4H), 1.70-1.59 (m, 1H), 1.35 s, 3H), 1.30-1.20 (m, 9H). ¹³C (75 MHz, CDCl₃) δ 197.9, 183.3, 154.0, 144.3, 140.1, 129.6, 126.1, 125.0, 46.5, 43.6, 37.8, 37.5, 37.1, 36.6, 30.1, 23.7, 22.7, 22.6, 18.1, 16.3.

### Synthesis of Wu21

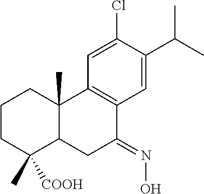 [SMILES: CC(C)c1cc2c(cc1Cl)[C@@]1(C)CCC[C@@](C)(C(=O)O)C1C/C2=N\O]

(1R,4aS,E)-6-chloro-7-isopropyl-9-hydroxyimino-1,4a-dimethyl-1,2,3,4,4a,9,10,10a-octahydrophenanthrene-1-carboxylic acid (Wu21): Followed the general procedure B, ketone Wu20 (14.3 mg, 0.041 mMol), hydroxylamine hydrochloride (5.7 mg, 0.082 mMol) and pyridine (6.8 mg, 0.086 mMol) were used and 9.0 mg product Wu21 (60% yield) was achieved. NMR ¹H (300 MHz, CDCl₃) δ 7.68 (s, 1H), 7.24 (s, 1H), 3.30 (m, 1H), 2.92-2.63 (m, 2H), 2.32 (dd, J=12.9, 4.8 Hz, 1H), 2.25 (br d, J=12.3 Hz, 1H), 1.84-1.72 (m, 4H), 1.67-1.56 (m, 1H), 1.39 (s, 3H), 1.18 (d, J=6.9 Hz, 3H), 1.16 (d, J=6.6 Hz, 3H), 1.12 (s, 3H). ¹³C (75 MHz, CDCl₃) δ 183.2, 155.8, 150.6, 144.1, 136.4, 126.4, 124.6, 123.4, 46.1, 41.4, 37.1, 36.8, 30.1, 24.5, 22.9, 22.7, 22.6, 18.1, 16.6.

### Synthesis of Wu23

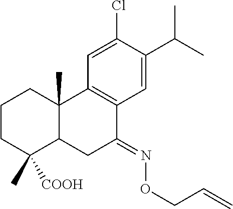 [SMILES: C=CCO/N=C1\CC2[C@](C)(C(=O)O)CCC[C@]2(C)c2cc(Cl)c(C(C)C)cc21]

(1R,4aS,E)-9-((allyloxy)imino)-6-chloro-7-isopropyl-1,4a-dimethyl-1,2,3,4,4a,9,10,10a-octahydrophenanthrene-1-carboxylic acid (Wu23): Followed the general procedure B, ketone Wu20 (35.4 mg, 0.101 mMol), 0-allylhydroxylamine hydrochloride (22.2 mg, 0.203 mMol) and pyridine (16.9 mg, 0.213 mMol) were used and 30.7 mg product Wu23 (75% yield) was achieved. NMR ¹H (500 MHz, CDCl₃) δ 7.81 (s, 1H), 7.21 (s, 1H), 6.15-6.01 (m, 1H), 5.34 (dd, J=17.0, 1.5 Hz, 1H), 5.24 (d, J=10.0 Hz, 1H), 4.74-4.65 (m, 2H), 3.33 (m, 1H), 2.71 (dd, J=19.0, 5.0 Hz, 1H), 2.61 (dd, J=19.0, 14.0 Hz, 1H), 2.40-2.19 (m, 2H), 1.80-1.55 (m, 5H), 1.36 (s, 3H), 1.27 (d, J=7.0 Hz, 3H), 1.24 (d, J=7.0 Hz, 3H), 1.11 (s, 3H). ¹³C (125 MHz, CDCl₃) δ 184.0, 153.5, 149.7, 143.5, 135.0, 134.7, 128.2, 122.9, 117.6, 75.5, 46.3, 41.5, 37.3, 37.1, 36.6, 30.2, 24.3, 22.9, 22.8, 22.7, 18.1, 16.5

### Synthesis of Wu24

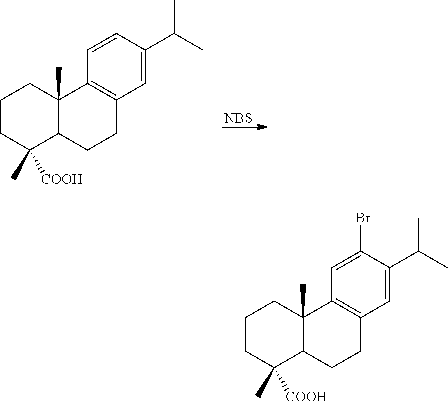 [SMILES: CC(C)c1ccc2c(c1)CCC1[C@](C)(C(=O)O)CCC[C@]21C ; "NBS" ; CC(C)c1cc2c(cc1Br)[C@@]1(C)CCC[C@@](C)(C(=O)O)C1CC2]

(1R,4aS,E)-6-bromo-7-isopropyl-1,4a-dimethyl-1,2,3,4,4a,9,10,10a-octahydrophenanthrene-1-carboxylic acid (Wu24): The mixture of dehydroabietic acid (400.0 mg, 1.0 mMol) and N-bromosuccinimide (189.6 mg, 0.8 mMol) in 3 mL acetonitrile in a 2.0-5.0 mL vial was heated under microwave irradiation at 90° C. for 30 min, the conversion was not completed according to HPLC, then another 189.6 mg of N-bromosuccinimide was added and stirred at 90° C. for another 30 min. Full conversion was not achieved either, 189.6 mg NBS was added again and heated under microwave at 90° C. for another 30 min, this time full conversion was achieved. Concentrated and purified on silica gel with 20-45% EA-n-heptane (0.1% HCOOH) to give 252.0 mg Wu24 (50% yield) as white solid. NMR ¹H (300 MHz, CDCl₃) δ 7.37 (s, 1H), 6.92, (s, 1H), 3.27 (m, 1H), 2.96-2.81 (m, 2H), 2.26 (br d, J=12.9 Hz, 1H), 2.19 (dd, J=12.3, 1.8 Hz, 1H), 1.84-1.68 (m, 6H), 1.60-1.48 (m, 1H), 1.28 (s, 3H), 1.26-1.17 (m, 9H). ¹³C (75 MHz, CDCl₃) δ 185.4, 148.9, 144.2, 134.6, 128.6, 127.3, 121.6, 47.5, 44.4, 37.9, 37.1, 36.8, 32.5, 29.6, 25.1, 23.1, 22.9, 21.7, 18.5, 16.3.

### Synthesis of Wu26

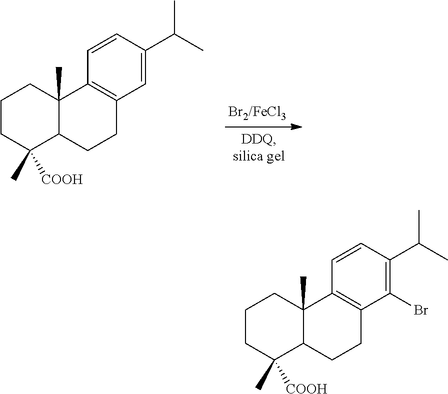 [SMILES: CC(C)c1ccc2c(c1)CCC1[C@](C)(C(=O)O)CCC[C@]21C ; CC(C)c1ccc2c(c1Br)CCC1[C@](C)(C(=O)O)CCC[C@]21C]

(1R,4aS)-8-bromo-7-isopropyl-1,4a-dimethyl-1,2,3,4,4a,9,10,10a-octahydrophenanthrene-1-carboxylic acid (Wu26): To the mixture of FeCl₃ (3.6 mg, 0.022 mMol), DDQ (2.8 mg, 0.016 mMol), and Merck silica gel 60 (220.0 mg) in DCM added dehydroabietic acid (400.0 mg, 1.332 mMol), then 41.2 uL Br₂ (127.8 mg, 0.8 mMol) was added at 0° C. and stirred at rt for about 40 min, about 25% conversed, then 82.4 uL Br₂ was added and stirred at rt, monitored with LC, full conversed was achieved after about 1 h at rt, concentrated and purified on silica gel with 25-45% EA-n-heptane (0.1% HCOOH) twice to give about 126.5 mg Wu26 (yield 25%) and 275.7 mg Wu24 (yield 55%). Wu26: NMR ¹H (300 MHz, CDCl₃) δ 7.21 (d, J=8.1 Hz, 1H), 7.09 (d, J=8.4 Hz, 1H), 3.45 (m, 1H), 3.00 (dd, J=18.0, 6.9 Hz, 1H), 2.90-2.74 (m, 1H), 2.31 (br d, J=12.9 Hz, 1H), 2.18 (dd, 12.3, 1.8 Hz, 1H), 1.90-1.50 (m, 7H), 1.29 (s, 3H), 1.27-1.18 (m, 9H). NMR ¹³C (75 MHz, CDCl₃) δ 185.0, 149.3, 145.0, 135.0, 128.0, 123.9, 123.5, 47.4, 43.9, 38.4, 37.4, 36.7, 33.1, 32.8, 25.2, 23.2, 22.9, 22.0, 18.7, 16.3.

### Synthesis of Wu33

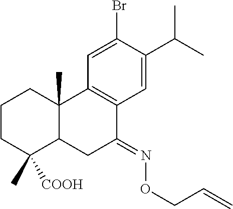 [SMILES: C=CCO/N=C1\CC2[C@](C)(C(=O)O)CCC[C@]2(C)c2cc(Br)c(C(C)C)cc21]

(1R,4aS,E)-6-bromo-9-((allyloxy)imino)-7-isopropyl-1,4a-dimethyl-1,2,3,4,4a,9,10,10a-octahydrophenanthrene-1-carboxylic acid (Wu33): Followed the general procedure B, ketone Wu30 (22.0 mg, 0.056 mMol), 0-allylhydroxylamine hydrochloride (12.3 mg, 0.112 mMol) and pyridine (9.3 mg, 0.118 mMol) were used and 15.7 mg product Wu33 (63% yield) was achieved. NMR ¹H (500 MHz, CDCl₃) δ 7.79 (s, 1H), 7.40 (s, 1H), 6.13-6.02 (m, 1H), 5.33 (d, J=17.0 Hz, 1H), 5.23 (d, J=11.0 Hz, 1H), 4.72 (d, J=6.0 Hz, 1H), 3.29 (m, 1H), 2.71 (dd, J=19.0, 5.0 Hz, 1H), 2.61 (dd, J=18.5, 13.5 Hz, 1H), 2.28-2.19 (m, 2H), 1.80-1.71 (m, 4H), 1.66-1.57 (m, 1H), 1.35 (s, 3H), 1.26 (d, J=7.0 Hz, 3H), 1.23 (d, J=7.0 Hz, 3H), 1.11 (s, 3H). ¹³C (75 MHz, CDCl₃) 5182.1, 153.5, 149.9, 134.7, 128.9, 127.5, 126.1, 122.9, 117.6, 75.6, 46.2, 41.6, 37.3, 37.2, 36.6, 32.8, 24.3, 23.0, 22.9, 18.1, 16.5.

### Synthesis of Wu52

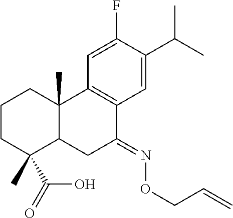 [SMILES: C=CCO/N=C1\CC2[C@](C)(C(=O)O)CCC[C@]2(C)c2cc(F)c(C(C)C)cc21]

(1R,4aS,E)-9-((allyloxy)imino)-6-fluoro-7-isopropyl-1,4a-dimethyl-1,2,3,4,4a,9,10,10a-octahydrophenanthrene-1-carboxylic acid (Wu52): Followed the general procedure B, Wu108 (8.7 mg, 0.027 mMol), 0-allylhydroxylamine hydrochloride (6.0 mg, 0.055 mMol) and pyridine (4.5 mg, 0.057 mMol) were used and 10.6 mg product Wu52 (100% yield) was achieved. NMR 1H (300 MHz, CDCl3) δ 7.78 (d, J=8.1 Hz), 6.87 (d, J=11.7 Hz, 1H), 6.17-6.00 (m, 1H), 5.33 (dd, J=17.7, 1.8 Hz, 1H), 5.23 (dd, J=11.7, 1.2 Hz, 1H), 4.70 (d, J=5.4 Hz, 2H), 3.16 (m, 1H), 2.73 (dd, J=18.9, 5.4 Hz, 1H), 2.60 (dd, J=18.6, 12.9 Hz, 1H), 2.27 (dd, J=12.9, 5.4 Hz, 1H), 2.19 (br d, J=13.5 Hz, 1H), 1.82-1.52 (m, 5H), 1.37 (s, 3H), 1.27 (d, J=6.6 Hz, 3H), 1.25 (d, J=6.3 Hz, 3H), 1.11 (s, 3H), 13C (75 MHz, CDCl₃) δ 182.2, 162.0 (d, J=246.2 Hz), 153.5, 150.7 (d, J=6.9 Hz), 134.8, 133.4, 125.3 (d, J=3.5 Hz), 124.1 (d, J=5.7 Hz), 117.5, 110.0 (d, J=24.1 Hz), 75.4, 46.3, 41.7, 37.3, 37.2, 36.6, 27.7, 24.3, 22.9, 22.6, 18.1, 16.5.

### Synthesis of Wu55

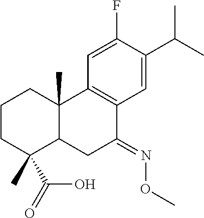 [SMILES: CO/N=C1\CC2[C@](C)(C(=O)O)CCC[C@]2(C)c2cc(F)c(C(C)C)cc21]

(1R,4aS,E)-6-fluoro-7-isopropyl-9-(methoxyimino)-1,4a-dimethyl-1,2,3,4,4a,9,10,10a-octahydrophenanthrene-1-carboxylic acid (Wu55): Followed the general procedure B, 12F-ketone (11.4 mg, 0.034 mMol), O-methylhydroxylamine hydrochloride (5.7 mg, 0.069 mMol) and pyridine (5.7 mg, 0.070 mMol) were used and 12.1 mg product Wu55 (98% yield) was achieved. NMR 1H (300 MHz, CDCl₃) δ 7.79 (d, J=8.1 Hz), 6.87 (d, J=12.3 Hz, 1H), 4.00 (s, 3H), 3.17 (m, 1H), 2.75-2.52 (m, 2H), 2.20-2.14 (m, 2H), 1.82-1.71 (m, 4H), 1.65-1.54 (m, 1H), 1.36 (s, 3H), 1.27 (d, J=6.6 Hz, 3H), 1.26 (d, J=6.9 Hz, 3H), 1.10 (s, 3H), 13C (75 MHz, CDCl₃) δ 182.6, 162.0 (d, J=248.7 Hz), 153.4, 150.7 (d, J=6.9 Hz), 133.3 (d, J=16.1 Hz), 125.2 (d, J=3.4 Hz), 124.0 (d, J=5.8 Hz), 110.0 (d, J=24.0 Hz), 62.2, 46.3, 41.6, 37.3, 37.1, 36.6, 27.6, 24.2, 22.8, 22.7, 22.6, 18.1, 16.5.

### Synthesis of Wu60 and Wu61

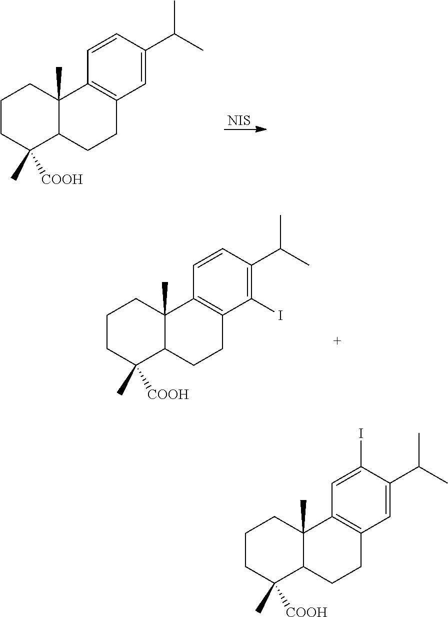 [SMILES: CC(C)c1ccc2c(c1)CCC1[C@](C)(C(=O)O)CCC[C@]21C ; "NIS" ; CC(C)c1ccc2c(c1I)CCC1[C@](C)(C(=O)O)CCC[C@]21C ; CC(C)c1cc2c(cc1I)[C@@]1(C)CCC[C@@](C)(C(=O)O)C1CC2]

(1R,4aS,E)-8-iodo-7-isopropyl-1,4a-dimethyl-1,2,3,4,4a,9,10,10a-octahydrophenanthrene-1-carboxylic acid (Wu60) and (1R,4aS,E)-6-iodo-7-isopropyl-1,4a-dimethyl-1,2,3,4,4a,9,10,10a-octahydrophenanthrene-1-carboxylic acid (Wu61): The mixture of dehydroabietic acid (500.0 mg, 1.664 mMol), N-iodosuccinimide (243.0 mg, 1.080 mMol) and 0.76 mL TFA (1.130 g, 11.648 mMol) in 10 mL acetonitrile in a 10.0-20.0 mL vial was heated under microwave irradiation at 90° C. for 60 min, then another part of N-iodosuccinimide (218.0 mg, 0.969 mMol) was added and irradiated again under microwave for another 60 min at 90° C. Full conversion was achieved and the crude HNMR was recorded, the ratio of the 14IDHAA and 12IDHAA was about 0.2:1 according to HNMR. Concentrated and purified on silica gel with 20-40% EA-n-heptane (0.1% HCOOH) and with preparative HPLC (70%-90% acetonitrile in water, 10 M NH40Ac) to give 56.6 mg Wu61 (yield 8%) and 345.6 mg Wu60 (yield 55%).

Wu60: NMR ¹H (300 MHz, CDCl₃) δ 7.64 (s, 1H), 6.89 (s, 1H), 3.09 (m, 1H), 2.91-2.82 (m, 2H), 2.25 (br d, J=12.9 Hz, 1H), 2.19 (dd, J=12.3, 2.4 Hz, 1H), 1.90-1.70 (m, 5H), 1.60-1.45 (m, 2H), 1.28 (s, 3H), 1.24-1.16 (m, 9H). ¹³C (75 MHz, CDCl₃) δ 185.4, 149.2, 147.3, 135.6, 135.5, 126.6, 98.2, 47.4, 44.4, 37.9, 37.6, 36.9, 36.8, 29.7, 25.2, 23.4, 23.2, 21.6, 18.5, 16.3.

Wu61: NMR ¹H (300 MHz, CDCl₃) δ 7.25 (d, J=8.4 Hz, 1H), 7.03 (d, J=8.4 Hz, 1H), 3.42-3.30 (m, 1H), 2.99-2.66 (m, 2H), 2.30 (d, J=12.9 Hz, 1H), 2.17 (d, J=12.3 Hz), 1.95-1.40 (m, 7H), 1.29 (s, 3H), 1.25-1.16 (m, 9H). ¹³C (75 MHz, CDCl₃) δ 184.5, 149.2, 148.6, 137.7, 124.7, 123.3, 111.3, 47.4, 44.1, 39.6, 38.8, 38.5, 37.5, 36.7, 25.3, 23.5, 23.2, 22.8, 18.8, 16.4.

Synthesis of compound Wu62

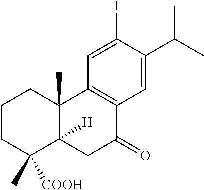

(1R,4aS,10aR)-6-iodo-7-isopropyl-9-oxo-1,4a-dimethyl-1,2,3,4,4a,9,10,10a-octahydrophenanthrene-1-carboxylic acid (Wu62): Followed the general procedure A, compound Wu60 (300.0 mg, 0.704 mMol) and CrO₃ (98.5 mg, 0.985 mMol) were used as started materials, the mixture in HOAc was heated at 50° C. for 3 h, then overnight at rt. Concentrated and purified on silica gel to give compound Wu62 (205.1 mg, yield 66%). NMR ¹H (300 MHz, CDCl₃) δ 7.822 (s, 1H), 7.817 (s, 1H), 3.15 (m, 1H), 2.80-2.60 (m, 2H), 2.48 (d, J=14.1 Hz, 1H), 2.30 (d, J=12.3 Hz, 1H), 1.85-1.70 (m, 4H), 1.70-1.55 (m, 1H), 1.33 (s, 3H), 1.26 (s, 3H), 1.30-1.18 (m, 9H). ¹³C (75 MHz, CDCl₃) δ 198.4, 183.2, 154.0, 149.1, 135.3, 131.1, 124.6, 109.9, 46.5, 43.6, 38.0, 37.8, 37.3, 37.1, 36.6, 23.7, 23.2, 23.0, 18.1, 16.3.

### Synthesis of Wu63

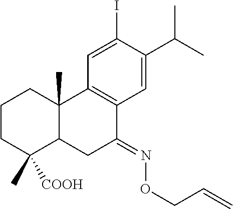 [SMILES: C=CCO/N=C1\CC2[C@](C)(C(=O)O)CCC[C@]2(C)c2cc(I)c(C(C)C)cc21]

(1R,4aS,E)-9-((allyloxy)imino)-6-iodo-7-isopropyl-1,4a-dimethyl-1,2,3,4,4a,9,10,10a-octahydrophenanthrene-1-carboxylic acid (Wu63): Followed the general procedure B, ketone Wu62 (29.5 mg, 0.067 mMol), 0-allylhydroxylamine hydrochloride (14.7 mg, 0.134 mMol) and pyridine (11.1 mg, 0.141 mMol) were used and 28.3 mg product Wu63 (85% yield) was achieved. NMR ¹H (300 MHz, CDCl₃) δ 7.74 (s, 1H), 7.67 (s, 1H), 6.18-6.01 (m, 1H), 5.33 (dd, J=17.1, 1.8 Hz, 1H), 5.23 (dd, J=10.5, 1.5 Hz, 1H), 4.75-4.69 (m, 2H), 3.12 (m, 1H), 2.78-2.55 (m, 2H), 2.30-2.18 (m, 2H), 1.81-1.53 (m, 5H), 1.35 (s, 3H), 1.25 (d, J=6.3 Hz, 3H), 1.22 (d, J=6.6 Hz, 3H), 1.11 (s, 3H). ¹³C (75 MHz, CDCl₃) δ 183.6, 153.7, 150.2, 148.2, 134.7, 134.4, 129.8, 122.0, 117.6, 103.4, 75.6, 46.3, 41.4, 37.9, 37.3, 37.1, 36.4, 24.3, 23.3, 23.1, 23.0, 18.1, 16.5.

### Synthesis of Wu64

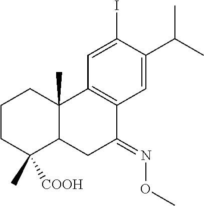 [SMILES: CO/N=C1\CC2[C@](C)(C(=O)O)CCC[C@]2(C)c2cc(I)c(C(C)C)cc21]

(1R,4aS,E)-6-iodo-7-isopropyl-9-(methoxyimino)-1,4a-dimethyl-1,2,3,4,4a,9,10,10a-octahydrophenanthrene-1-carboxylic acid (Wu64): Followed the general procedure B, ketone Wu62 (20.0 mg, 0.045 mMol), O-methylhydroxylamine hydrochloride (7.6 mg, 0.091 mMol) and pyridine (7.5 mg, 0.095 mMol) were used and 16.3 mg product Wu64 (76% yield) was achieved. NMR ¹H (300 MHz, CDCl₃) δ 7.75 (s, 1H), 7.67 (s, 1H), 4.01 (s, 3H), 3.12 (m, 1H), 2.70-2.50 (m, 2H), 2.28-2.17 (m, 2H), 1.80-1.70 (m, 4H), 1.66-1.55 (m, 1H), 1.35 (s, 3H), 1.26 (d, J=6.9 Hz, 3H), 1.23 (d, J=6.9 Hz, 3H), 1.10 (s, 3H). ¹³C (75 MHz, CDCl₃) δ 183.4, 153.5, 150.1, 148.3, 134.4, 129.7, 121.9, 103.4, 62.3, 46.3, 41.4, 38.0, 37.3, 37.1, 36.4, 24.2, 23.3, 23.2, 22.9, 18.1, 16.5.

Synthesis of compound Wu68

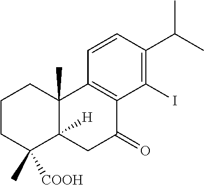

(1R,4aS,10aR)-8-iodo-7-isopropyl-1,4a-dimethyl-9-oxo-1,2,3,4,4a,9,10,10a-octahydrophenanthrene-1-carboxylic acid (Wu68): Followed the general procedure A, compound Wu61 (48.2 mg, 0.113 mMol) and CrO₃ (15.8 mg, 0.158 mMol) were used as started materials, then the mixture in HOAc was heated at 50° C. for 3 h, then overnight at rt. Concentrated and purified on silica gel to give compound Wu68 (6.4 mg, yield 13%). NMR ¹H (300 MHz, CDCl₃) δ 7.32 (d, J=8.1 Hz, 1H), 7.25 (d, J=8.1 Hz, 1H), 3.54 (m, 1H), 2.82-2.55 (m, 3H), 2.25 (d, J=11.7 Hz, 1H), 1.82-1.56 (m, 5H), 1.35 (s, 3H), 1.23 (d, J=6.6 Hz, 3H), 1.19 (d, J=6.9 Hz, 3H), 1.16 (s, 3H). ¹³C (75 MHz, CDCl₃) 5199.3, 183.0, 154.4, 151.1, 135.4, 129.9, 123.2, 100.2, 45.8, 42.0, 38.3, 37.7, 37.5, 36.8, 23.4, 23.2, 23.1, 18.1, 16.6.

### Synthesis of Wu69

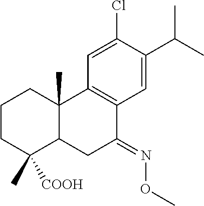 [SMILES: CO/N=C1\CC2[C@](C)(C(=O)O)CCC[C@]2(C)c2cc(Cl)c(C(C)C)cc21]

(1R,4aS,E)-6-chloro-7-isopropyl-9-methoxyimino-1,4a-dimethyl-1,2,3,4,4a,9,10,10a-octahydrophenanthrene-1-carboxylic acid (Wu69): Followed the general procedure B, ketone Wu20 (21.3 mg, 0.061 mMol), hydroxylamine hydrochloride (10.2 mg, 0.122 mMol) and pyridine (10.1 mg, 0.128 mMol) were used and 22.3 mg product Wu69 (97% yield) was achieved. NMR ¹H (300 MHz, CDCl₃) δ 7.83 (s, 1H), 7.21 (s, 1H), 4.01 (s, 3H), 3.34 (m, 1H), 2.69 (dd, J=18.6, 5.4 Hz, 1H), 2.58 (dd, J=18.6, 12.9 Hz, 1H), 2.30-2.19 (m, 2H), 1.80-1.53 (m, 5H), 1.36 (s, 3H), 1.31-1.20 (m, 6H), 1.11 (s, 3H). ¹³C (75 MHz, CDCl₃) δ 183.5, 153.3, 149.7, 143.5, 135.0, 128.1, 124.2, 122.9, 62.3, 46.3, 41.5, 37.3, 37.1, 36.6, 30.2, 24.2, 22.9, 22.8, 22.7, 18.1, 16.5.

### Synthesis of Wu74

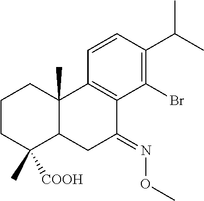 [SMILES: CO/N=C1\CC2[C@](C)(C(=O)O)CCC[C@]2(C)c2ccc(C(C)C)c(Br)c21]

(1R,4aS,E)-8-bromo-7-isopropyl-9-(methoxyimino)-1,4a-dimethyl-1,2,3,4,4a,9,10,10a-octahydrophenanthrene-1-carboxylic acid, followed the general procedure B, ketone Wu73 (20.0 mg, 0.051 mMol), O-methylhydroxylamine hydrochloride (8.5 mg, 0.102 mMol) and pyridine (8.9 mg, 0.112 mMol) were used and 17.0 mg product Wu74 (79% yield) was achieved. NMR ¹H (500 MHz, CDCl₃) δ 7.21 (d, J=8.5 Hz, 1H), 7.16 (d, J=9.0 Hz, 1H), 4.05 (s, 3H), 3.57 (m, 1H), 3.05 (dd, J=18.5, 13.0 Hz, 1H), 2.43 (dd, J=18.5, 6.5 Hz, 1H), 2.21 (br d, J=12.5 Hz, 1H), 2.13 (J=13.0, 6.5 Hz, 1H), 1.81-1.70 (m, 4H), 1.65-1.55 (m, 1H), 1.39 (s, 3H), 1.25 (d, J=7.0 Hz, 3H), 1.19 (d, J=6.5 Hz, 3H), 1.08 (s, 3H). NMR ¹³C (75 MHz, CDCl₃) δ 183.6, 153.7, 151.5, 147.3, 130.5, 127.0, 122.6, 121.7, 62.4, 46.1, 41.4, 37.8, 37.2, 33.4, 24.8, 23.5, 22.9, 21.4, 18.1, 16.7.

### Synthesis of Wu78

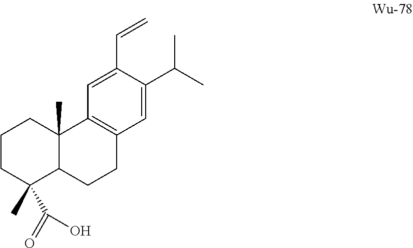 [SMILES: C=Cc1cc2c(cc1C(C)C)CCC1[C@](C)(C(=O)O)CCC[C@]21C]

(1R,4aS)-7-isopropyl-1,4a-dimethyl-6-vinyl-1,2,3,4,4a,9,10,10a-octahydrophenanthrene-1-carboxylic acid (Wu78) mMol) in 0.8 mL solvent mixture of DME/water (3:1) was irradiated under MW for 20 min at 140° C. Water was added, adjusted PH to 4, extracted with EA 3 ml×3. Concentrated and purified on PHPLC (45-90% CH₃CN in water, 10 mM NH₄OAc) to give 4.4 mg Wu78, yield 32% for both isomers. Wu78: ¹H NMR (500 MHz, CDCl₃) δ 7.32 (s, 1H), 7.03 (dd, J=17.0, 11.0 Hz, 1H), 6.91 (s, 1H), 5.53 (dd, J=17.0, 1.5 Hz, 1H), 5.22 (dd, J=11.0, 1.5 Hz, 1H), 3.16 (m, 1H), 2.95-2.80 (m, 2H), 2.37 (d, J=12.0 Hz, 1H), 2.24 (d, J=11.5 Hz, 1H), 1.90-1.65 (m, 5H), 1.60-1.50 (m, 2H), 1.29 (s, 3H), 1.24 (s, 3H), 1.22 (d, J=7.0 Hz, 3H), 1.19 (d, J=6.5 Hz, 3H). ¹³C NMR (75 MHz, CDCl₃) δ 184.0, 146.9, 143.0, 135.5, 135.0, 133.9, 125.5, 122.0, 114.7, 47.5, 44.8, 38.0, 37.1, 36.9, 30.0, 28.8, 25.3, 23.7, 23.4, 21.9, 18.7, 16.5.

### Synthesis of Wu86

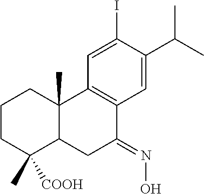 [SMILES: CC(C)c1cc2c(cc1I)[C@@]1(C)CCC[C@@](C)(C(=O)O)C1C/C2=N\O]

(1R,4aS,E)-9-(hydroxyimino)-6-iodo-7-isopropyl-1,4a-dimethyl-1,2,3,4,4a,9,10,10a-octahydrophenanthrene-1-carboxylic acid (Wu86): Followed the general procedure B, ketone Wu62 (20.5 mg, 0.047 mMol), hydroxylamine hydrochloride (6.5 mg, 0.093 mMol) and pyridine (7.7 mg, 0.112 mMol) were used and 20.5 mg product Wu86 (97% yield) was achieved. NMR 1H (500 MHz, CDCl₃) δ 7.70 (s, 1H), 7.52 (s, 1H), 3.08 (m, 1H), 2.83 (dd, J=19.0, 5.5 Hz, 1H), 2.69 (dd, J=19.0, 14.0 Hz, 1H), 2.31 (dd, J=14.0, 5.5 Hz, 1H), 2.24 (br d, J=12.5 Hz, 1H), 1.82-1.68 (m, 4H), 1.65-1.55 (m, 1H), 1.39 (s, 3H), 1.18-1.08 (m, 9H), 13C (125 MHz, CDCl₃) δ 183.3, 155.1, 150.7, 148.6, 134.7, 129.0, 121.8, 104.0, 46.1, 41.5, 37.9, 37.3, 37.2, 36.6, 24.2, 23.2, 23.0, 22.9, 18.1, 16.6.

Synthesis of compound Wu90

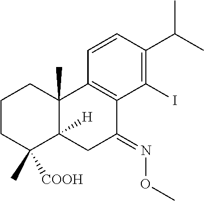

(1R,4aS,10aR)−)-8-iodo-7-isopropyl-9-methoxyimino-1,4a-dimethyl-1,2,3,4,4a,9,10,10a-octahydrophenanthrene-1-carboxylic acid (Wu90): Followed the general procedure B, ketone 64 (8.8 mg, 0.020 mMol), O-methylhydroxylamine hydrochloride (3.3 mg, 0.040 mMol) and pyridine (3.3 mg, 0.042 mMol) were used and product Wu90 (4.5 mg, 51% yield) was achieved. NMR ¹H (300 MHz, CDCl₃) δ 7.19 (d, J=8.1 Hz, 1H), 7.15 (d, J=8.1 Hz, 1H), 4.07 (s, 3H), 3.45 (m, 1H), 3.08 (dd, J=18.3, 12.9 Hz, 1H), 2.42 (dd, J=18.3, 6.3 Hz, 1H), 2.21 (br d, J=12.3 Hz, 1H), 2.14 (dd, J=12.9, 6.3 Hz, 1H), 1.80-1.53 (m, 5H), 1.39 (s, 3H), 1.24 (d, J=6.3 Hz, 3H), 1.19 (d, J=7.2 Hz, 3H), 1.07 (s, 3H). ¹³C (75 MHz, CDCl₃) δ 182.3, 155.2, 150.9, 150.7, 134.0, 126.7, 122.7, 101.5, 62.3 46.0, 41.5, 39.3, 37.70, 37.65, 37.1, 24.5, 23.8, 23.2, 21.3, 18.0, 16.7. HRMS calculated mass: 470.1192 [M+H], measured: 470.1186 [M+H].

Synthesis of compound Wu91

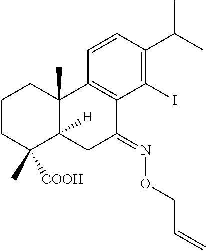

(1R,4aS,10aR)-9-((allyloxy)imino)-8-iodo-7-isopropyl-1,4a-dimethyl-1,2,3,4,4a,9,10,10a-octahydrophenanthrene-1-carboxylic acid (Wu91): Followed the general procedure B, ketone 64 (6.0 mg, 0.014 mMol), 0-Allylhydroxylamine hydrochloride (4.5 mg, 0.041 mMol) and pyridine (3.3 mg, 0.042 mMol) were used and compound Wu91 (5.9 mg, 88% yield) was achieved. NMR ¹H (300 MHz, CDCl₃) δ 7.19 (d, J=8.1 Hz, 1H), 7.15 (d, J=8.1 Hz, 1H), 6.25-6.08 (m, 1H), 5.36 (dd, J=17.1, 1.8 Hz, 1H), 5.24 (dd, J=10.5, 1.8 Hz, 1H), 4.82-4.68 (m, 2H), 3.47 (m, 1H), 3.13 (dd, J=18.0, 12.9 Hz, 1H), 2.41 (dd, J=18.0, 6.3 Hz, 1H), 2.25-2.09 (m, 2H), 1.82-1.70 (m, 4H), 1.66-1.57 (m, 1H), 1.39 (s, 3H), 1.24 (d, J=7.2 Hz, 3H), 1.18 (d, J=6.9 Hz, 3H), 1.07 (s, 3H). ¹³C (75 MHz, CDCl₃) δ 182.9, 155.6, 150.9, 150.7, 135.4, 134.2, 126.6, 122.6, 117.5, 101.4, 75.4, 46.0, 41.5, 39.3, 37.7, 37.2, 24.7, 23.8, 23.2, 21.4, 18.0, 16.7. HRMS calculated mass: 496.1349 [M+H], measured: 496.1342 [M+H].

Synthesis of compound Wu104

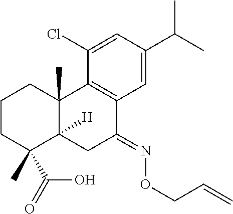

(1R,4aS,10aR)-9-((allyloxy)imino)-5-chloro-7-isopropyl-1,4a-dimethyl-1,2,3,4,4a,9,10,10a-octahydrophenanthrene-1-carboxylic acid (Wu104): Followed the general procedure B, (1R,4aS,10aR)-5-chloro-7-isopropyl-1,4a-dimethyl-9-oxo-1,2,3,4,4a,9,10,10a-octahydrophenanthrene-1-carboxylic acid (8.0 mg, 0.023 mMol), 0-allylhydroxylamine hydrochloride (12.6 mg, 0.115 mMol) and pyridine (9.2 mg, 0.117 mMol) were used and compound Wu104 (2.2 mg, 24% yield) was achieved. NMR ¹H (300 MHz, CDCl₃) δ 7.76 (d, J=1.8 Hz, 1H), 7.17 (d, J=1.8 Hz, 1H), 6.12-5.97 (m, 1H), 5.35-5.26 (m, 1H), 5.26-5.16 (m, 1H), 4.72-4.65 (m, 2H), 3.43 (br d, J=13.8 Hz, 1H), 2.85 (m, 1H), 2.77 (dd, J=17.4, 3.6 Hz, 1H), 2.50 (dd, J=17.4, 13.8 Hz, 1H), 2.34 (dd, J=13.8, 3.6 Hz, 1H), 1.80-1.67 (m, 4H), 1.58-1.46 (m, 1H), 1.36 (s, 3H), 1.33 (s, 3H), 1.23 (d, J=6.9 Hz, 3H), 1.22 (d, J=6.9 Hz, 3H). ¹³C (75 MHz, CDCl₃) δ 182.1, 154.8, 147.8, 143.6, 134.7, 132.8, 132.8, 132.7, 122.2, 117.5, 75.5, 47.2, 42.7, 40.2, 36.8, 33.3, 24.0, 23.9, 23.5, 18.8, 18.4, 16.7. HRMS calculated mass: 404.1993 [M+H], 406.1967 [M+H+2], measured: 404.1987 [M+H], 406.1968 [M+H+2].

Synthesis of compound Wu105

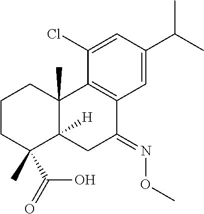

(1R,4aS,10aR)-5-chloro-7-isopropyl-9-(methoxyimino)-1,4a-dimethyl-1,2,3,4,4a,9,10,10a-octahydrophenanthrene-1-carboxylic acid Wu105: Followed the general procedure B, (1R,4aS,10aR)-5-chloro-7-isopropyl-1,4a-dimethyl-9-oxo-1,2,3,4,4a,9,10,10a-octahydrophenanthrene-1-carboxylic acid (16.9 mg, 0.048 mMol), O-methylhydroxylamine hydrochloride (16.2 mg, 0.194 mMol) and pyridine (16.8 mg, 0.213 mMol) were used and compound Wu105 (6.1 mg, 33% yield) was achieved. NMR ¹H (300 MHz, CDCl₃) δ 7.77 (d, J=1.8 Hz, 1H), 7.18 (d, J=1.8 Hz, 1H), 3.99 (s, 3H), 3.48 (br d, J=12.9 Hz, 1H), 2.85 (m, 1H), 2.71 (dd, J=18.0, 3.9 Hz, 1H), 2.54-2.42 (m, 1H), 2.32 (dd, J=14.1, 3.3 Hz, 1H), 1.80-1.66 (m, 4H), 1.58-1.48 (m, 1H), 1.35 (s, 3H), 1.32 (s, 3H), 1.24 (d, J=6.6 Hz, 3H), 1.23 (d, J=6.3 Hz, 3H). ¹³C (75 MHz, CDCl₃) δ 183.0, 154.6, 147.8, 143.6, 132.7, 131.8, 131.7, 122.1, 62.3, 47.3, 42.6, 40.2, 36.8, 36.3, 33.3, 23.9, 23.5, 18.8, 18.4, 16.7. HRMS calculated mass: 378.1836 [M+H], 380.1806 [M+H+2], measured: 378.1831 [M+H], 378.1810 [M+H+2].

### Synthesis of Compounds 25 (=Wu108) and 47 (=Wu56)

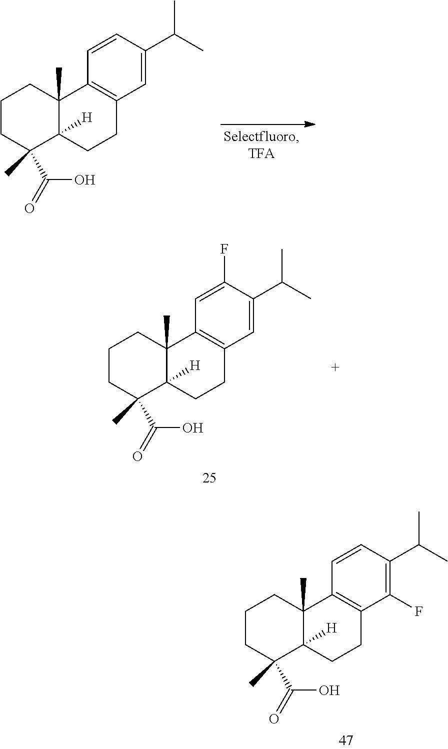

Supplementary Scheme 9. Synthesis of compounds 25 (=Wu108) and 47 (Wu56). (1R,4aS,10aR)-6-fluoro-7-isopropyl-1,4a-dimethyl-1,2,3,4,4a,9,10,10a-octahydrophenanthrene-1-carboxylic acid (Wu108) and (1R,4aS,10aR)-8-fluoro-7-isopropyl-1,4a-dimethyl-1,2,3,4,4a,9,10,10a-octahydrophenanthrene-1-carboxylic acid (Wu56): The reaction mixture of DHAA (60.0 mg, 0.166 mMol) and select fluoro Chloromethyl-4-fluoro-1,4-diazoniabicyclo[2.2.2]octane bis(tetrafluoroborate) (141.5 mg, 0.399 mMol) in 1.2 mL TFA was heated at 100° C. for 2 h under microwave irradiation. Three parallel reactions were run instead of running at 180 mg scale because it gave poorer yield at large scale, combined all the reaction mixtures, concentrated and dissolved in DCM, filtered to get rid of the insoluble side product, purified on silica gel with EtOAc/n-heptane/HCOOH (20:80:0.1 to 45:55:0.1), and then further purified with preparative HPLC (35-100% acetonitrile-water-10 mM NH₄OAc) to give Wu108 (6.4 mg, yield 3%) and Wu56 (12.5 mg, yield 7%). Wu108: NMR ¹H (300 MHz, CDCl₃) δ 6.90-6.80 (m, 2H), 3.13 (m, 1H), 2.92-2.79 (m, 2H), 2.26-2.14 (m, 2H), 1.86-1.68 (m, 5H), 1.60-1.51 (m, 2H), 1.28 (s, 3H), 1.26-1.18 (m, 9H). ¹³C (75 MHz, CDCl₃) δ 183.8, 159.3 (d, JcF=240 Hz), 148.6 (d, JcF=5.7 Hz), 132.4 (d, JcF=16.1 Hz), 127.6 (d, JcF=5.7 Hz), 124.2 (d, JcF=16.1 Hz), 110.8 (d, JCF=22.9 Hz), 47.4, 44.6, 38.1, 37.8, 29.4, 27.2, 25.1, 24.1, 22.9, 22.7, 21.9, 18.6, 16.4. ¹⁹F (282.2 MHz, CDCl₃) δ−124.3 (dd, J=11.9, 8.4 Hz). HRMS calculated mass: 317.1917 [M−H], measured: 317.1932 [M−H].

### Synthesis of Wu115, Wu50, Wu133 and Wu45

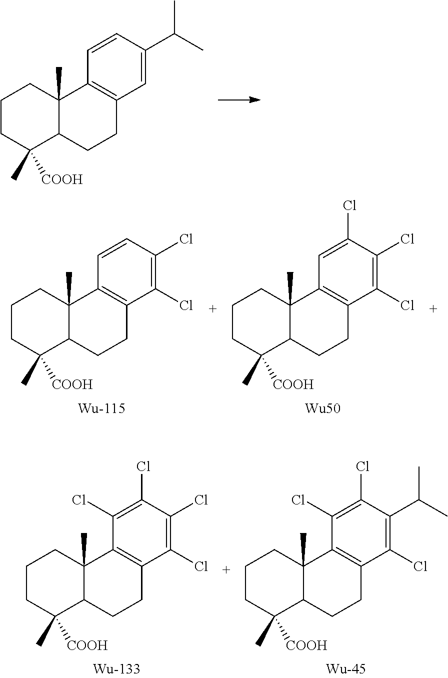

(1R,4aS)-7,8-dichloro-1,4a-dimethyl-1,2,3,4,4a,9,10,10a-octahydrophenanthrene-1-carboxylic acid (Wu115). To the mixture of dehydroabietic acid (200 mg, 0.666 mMol), FeCl₃ (49.7 mg, 0.306 mMol), DDQ and silica gel (12 mg) added 10 mL Cl₂ in CCl₄ at 0° C. and stirred at this temperature for about 2.5 h, The temperature was allowed to warm up rt and stirred overnight. The excess amount of Cl₂ was flushed away with air and concentrated, purified on silica gel with EtOAc/n-heptane/HCOOH (25:75:0.1 to 50:50:0.1) to give Wu45 (153.0 mg, 57% yield), and 50.5 mg mixture, which was purified further with preparative HPLC to give 4.1 mg Wu115 (yield 2%), Wu50 (16.2 mg, 7%) and Wu133 (5.6 mg, 4%). Wu45: NMR ¹H (300 MHz, CDCl₃) δ 4.03 (m, 1H), 3.55-3.41 (m, 1H), 2.90-2.80 (m, 2H), 2.09 (d, J=12.0 Hz, 1H), 1.80-1.56 (m, 6H), 1.51 (s, 3H), 1.41 (d, J=6.0 Hz, 6H), 1.32 (s, 3H), 1.28-1.15 (m, 1H).

Wu115: NMR ¹H (300 MHz, CDCl₃) δ 7.25 (d, J=8.7 Hz, 1H), 7.12 (d, J=8.7 Hz, 1H), 3.07-2.75 (m, 2H), 2.28 (d, J=1 2.3 Hz, 1H), 2.20-2.05 (m, 1H), 1.90-1.60 (m, 6H), 1.60-1.38 (m, 1H), 1.29 (s, 3H), 1.20 (s, 3H). ¹³C (75 MHz, CDCl₃) δ 183.7, 149.9, 135.7, 132.4, 130.0, 127.5, 123.6, 47.3, 43.7, 38.2, 37.4, 36.6, 29.4, 25.1, 21.4, 18.6, 16.4.

Wu133: NMR ¹H (300 MHz, CDCl₃) δ 3.52-3.43 (m, 1H), 2.94-2.84 (m, 2H), 2.10 (d, J=10.8 Hz, 1H), 1.82-1.55 (m, 6H), 1.49 (s, 3H), 1.32 (s, 3H), 1.29-1.17 (m, 1H). ¹³C (75 MHz, CDCl₃) δ 182.9, 147.0, 137.0, 133.2, 132.3, 130.7, 131.5, 48.2, 46.9, 41.3, 36.1, 35.0, 32.7, 21.4, 19.1, 18.7, 17.1.

### Synthesis of Wu119

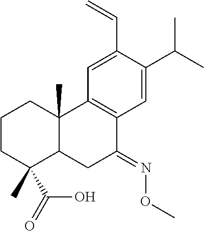 [SMILES: C=Cc1cc2c(cc1C(C)C)/C(=N/OC)CC1[C@](C)(C(=O)O)CCC[C@]21C]

(1R,4aS,E)-7-isopropyl-9-(methoxyimino)-1,4a-dimethyl-6-vinyl-1,2,3,4,4a,9,10,10a-octahydrophenanthrene-1-carboxylic acid (Wu119) The reaction mixture of Wu118 (17.3 mg, 0.0508 mMol), O-methyl-hydroxylamin hydrochlorid (17.0, mg, 0.203 mMol) and pyridine (16.8 uL, 0.208 mMol) in 1.0 mL EtOH was irradiated under MW at 110° C. for 1 h, purified on silica gel with EtOAc:nHeptane:HCOOH (30:70:0.5 to 50:50:0.5) to give 12.8 mg product, yield 68%. ¹H NMR (300 MHz, CDCl₃) δ 7.80 (s, 1H), 7.33 (s, 1H), 7.07 (dd, J=17.1, 10.8 Hz, 1H), 5.61 (dd, J=17.1, 1.5 Hz, 1H), 5.30 (dd, J=10.8, 1.5 Hz, 1H), 4.02 (s, 3H), 3.19 (m, 1H), 2.74-2.53 (m, 2H), 2.36-2.24 (m, 2H), 1.84-1.64 (m, 5H), 1.36 (s, 3H), 1.27 (d, J=7.2 Hz, 3H), 1.23 (d, J=7.2 Hz, 3H), 1.12 (s, 3H). ¹³C NMR (75 MHz, CDCl₃) δ 184.0, 154.0, 148.4, 143.7, 137.4, 135.2, 128.8, 121.1, 120.6, 115.9, 62.2, 46.4, 41.5, 37.3, 37.2, 36.5, 29.1, 24.3, 23.5, 23.4, 23.0, 18.2, 16.5.

### Synthesis of Wu120

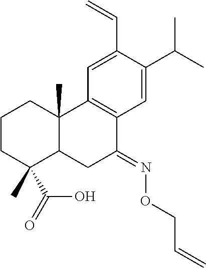 [SMILES: C=CCO/N=C1\CC2[C@](C)(C(=O)O)CCC[C@]2(C)c2cc(C=C)c(C(C)C)cc21]

(1R,4aS,E)-9-((allyloxy)imino)-7-isopropyl-1,4a-dimethyl-6-vinyl-1,2,3,4,4a,9,10,10a-octahydrophenanthrene-1-carboxylic acid (Wu120). The reaction mixture of Wu118 (17.6 mg, 0.0517 mMol), 0-allylhydroxylamine hydrochlorid (22.7 mg, 0.207 mMol) and pyridine (17.1 uL, 0.212 mMol) in 1.0 mL EtOH was irradiated under MW at 110° C. for 1 h, purified on silica gel with EtOAc:n-Heptane (25:75 to 40:60) to give 17.3 mg product, yield 91%. ¹H NMR (300 MHz, CDCl₃) δ 7.79 (s, 1H), 7.33 (s, 1H), 7.07 (dd, J=17.1, 10.8 Hz, 1H), 6.19-6.02 (m, 1H), 5.61 (dd, J=17.1, 1.8 Hz, 1H), 5.40-5.20 (m, 3H), 4.75-4.68 (m, 2H), 3.19 (m, 1H), 2.78-2.55 (m, 2H), 2.38-2.22 (m, 2H), 1.81-1.60 (m, 5H), 1.37 (s, 3H), 1.27 (d, J=7.2 Hz, 3H), 1.22 (d, J=7.2 Hz, 3H), 1.13 (s, 3H). ¹³C NMR (75 MHz, CDCl₃) δ 184.1, 154.1, 148.4, 143.7, 137.4, 135.2, 134.8, 128.9, 121.2, 120.6, 117.5, 115.9, 110.2, 75.5, 46.4, 41.5, 37.4, 37.2, 36.5, 29.1, 24.5, 23.5, 23.4, 23.1, 18.2, 16.5.

### Synthesis of Wu121

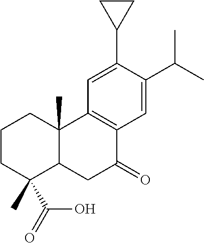 [SMILES: CC(C)c1cc2c(cc1C1CC1)[C@@]1(C)CCC[C@@](C)(C(=O)O)C1CC2=O]

(1R,4aS)-6-cyclopropyl-7-isopropyl-1,4a-dimethyl-9-oxo-1,2,3,4,4a,9,10,10a-octahydrophenanthrene-1-carboxylic acid (Wu121) The reaction mixture of 6-iodo-9-oxo-dehydroabietic acid (Wu-30) (84 mg, 0.191 mMol), cyclopropylboronic pinacolester (128.3 mg, 0.763 mMol), tetrakis (11.0 mg, 0.01 mMol) and Na₂CO₃ (101.2 mg, 0.955 mMol) in 3.2 mL solvent mixture of DME/water (3:1) was irradiated under MW for 30 min at 130° C. Water was added, adjusted PH to 4, extracted with EA 5 ml×3. Concentrated and purified on silica gel with EA:n-heptane:HCOOH (25:75 to 50:50), further purified on preparative LC (20-80% acetonitrile in water, 10 mM NH₄OAc) to give 14 mg product, yield 21%. ¹H NMR (300 MHz, CDCl₃) δ 7.89 (s, 1H), 6.92 (s, 1H), 3.48 (m, 1H), 2.80-2.62 (m, 2H), 2.44 (d, J=14.1 Hz, 1H), 2.34 (br d, J=12.6 Hz, 1H), 2.10-1.97 (m, 1H), 1.88-1.72 (m, 4H), 1.67-1.55 (m, 1H), 1.34 (s, 3H), 1.28 (d, J=6.9 Hz, 3H), 1.242 (d, J=6.9 Hz, 3H), 1.24 (s, 3H), 1.05-0.98 (m, 2H), 0.76-0.65 (m, 2H). ¹³C NMR (75 MHz, CDCl₃) δ 198.8, 183.1, 152.9, 147.4, 146.5, 128.8, 124.0, 120.4, 46.5, 43.8, 37.8, 37.4, 37.1, 36.6, 28.7, 23.8, 23.7, 23.5, 18.3, 16.3, 13.6, 8.3, 8.0.

### Synthesis of Wu122

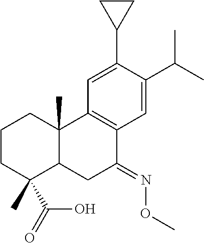 [SMILES: CO/N=C1\CC2[C@](C)(C(=O)O)CCC[C@]2(C)c2cc(C3CC3)c(C(C)C)cc21]

(1R,4aS,E)-6-cyclopropyl-7-isopropyl-9-(methoxyimino)-1,4a-dimethyl-1,2,3,4,4a,9,10,10a-octahydrophenanthrene-1-carboxylic acid (Wu122). The reaction mixture of Wu121 (10.1 mg, 0.0285 mMol), O-methyl-hydroxylamin hydrochlorid (9.5 mg, 0.114 mMol) and pyridine (9.4 uL, 0.117 mMol) in 1.0 mL EtOH was irradiated under MW at 110° C. for 1 h, purified on silica gel with EtOAc:nHeptane:HCOOH (30:70:0.5 to 50:50:0.5) to give 9.7 mg product, yield 89%. ¹H NMR (300 MHz, CDCl₃) δ 7.77 (s, 1H), 6.87 (s, 1H), 4.00 (s, 3H), 3.50 (m, 1H), 2.70-2.50 (m, 2H), 2.30-2.21 (m, 2H), 2.05-1.93 (m, 1H), 1.80-1.70 (m, 4H), 1.67-1.55 (m, 1H), 1.35 (s, 3H), 1.29 (d, J=6.9 Hz, 3H), 1.26 (d, J=6.9 Hz, 3H), 1.08 (s, 3H), 0.96-0.85 (m, 2H), 0.68-0.60 (m, 2H). ¹³C NMR (75 MHz, CDCl₃) δ 183.9, 154.1, 148.1, 146.2, 141.6, 127.0, 120.8, 120.4, 62.1, 46.4, 41.6, 37.3, 37.2, 36.5, 28.7, 24.3, 23.8, 23.7, 23.1, 18.2, 16.5, 13.4, 7.6, 7.2.

### Synthesis of Wu123

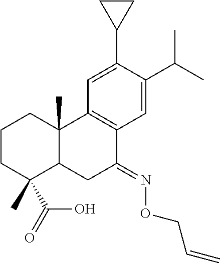 [SMILES: C=CCO/N=C1\CC2[C@](C)(C(=O)O)CCC[C@]2(C)c2cc(C3CC3)c(C(C)C)cc21]

(1R,4aS,E)-9-((allyloxy)imino)-6-cyclopropyl-7-isopropyl-1,4a-dimethyl-1,2,3,4,4a,9,10,10a-octahydrophenanthrene-1-carboxylic acid (Wu123) The reaction mixture of Wu121 (9.1 mg, 0.0257 mMol), 0-allyl-hydroxylamin hydrochlorid (11.2 mg, 0.114 mMol) and pyridine (8.5 uL, 0.105 mMol) in 1.0 mL EtOH was irradiated under MW at 110° C. for 1 h, purified on silica gel with EtOAc:nHeptane:HCOOH (30:70:0.5 to 50:50:0.5) to give 10.2 mg product, yield 97%. ¹H NMR (300 MHz, CDCl₃) δ 7.76 (s, 1H), 6.87 (s, 1H), 6.20-6.00 (m, 1H), 5.33 (dd, J=17.1, 1.8 Hz, 1H), 5.22 (dd, J=10.5, 1.8 Hz, 1H), 4.75-4.62 (m, 2H), 3.49 (m, 1H), 2.75-2.58 (m, 2H), 2.30-2.23 (m, 2H), 2.02-1.91 (m, 1H), 1.80-1.70 (m, 4H), 1.67-1.54 (m, 1H), 1.35 (s, 3H), 1.09 (s, 3H), 1.29 (d, J=6.9 Hz, 3H), 1.23 (d, J=7.2 Hz, 3H), 0.95-0.88 (m, 2H), 0.67-0.60 (m, 2H). ¹³C NMR (75 MHz, CDCl₃) δ 183.8, 154.3, 148.1, 146.1, 141.6, 134.9, 127.1, 120.9, 120.4, 117.4, 75.4, 46.4, 41.6, 37.4, 37.2, 36.5, 28.7, 24.5, 23.8, 23.7, 23.1, 18.2, 16.5, 13.4, 7.5, 7.3.

### Synthesis of Wu133

(1R,4aS)-5,6,7,8-tetrachloro-1,4a-dimethyl-1,2,3,4,4a,9,10,10a-octahydrophenanthrene-1-carboxylic acid (Wu133). See synthesis of Wu115.

### Synthesis of Wu134

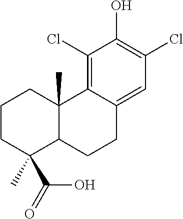 [SMILES: C[C@]1(C(=O)O)CCC[C@]2(C)c3c(cc(Cl)c(O)c3Cl)CCC12]

(1S,4aS)-5,7-dichloro-6-hydroxy-1,4a-dimethyl-1,2,3,4,4a,9,10,10a-octahydrophenanthrene-1-carboxylic acid (Wu134)

The mixture of podocarpic acid (PoCA) (40 mg, 0.146 mMol) and NCS (39.9 mg, 0.299 mMol) in 0.9 mL acetonitrile was irradiated under microwave at 100° C. for 1 h, conversion was not fully, another 8 mg of NCS was added and irradiated under microwave at 100° C. for another 30 min, then purified directly with preparative LC (20-90% acetonitrile in water, 10 mM NH₄OAc)) to give about 18.9 mg Wu-134 (38% yield). ¹H NMR (300 MHz, d6-acetone) δ 7.07 (s, 1H), 3.46-3.35 (m, 1H), 2.83-2.74 (m, 2H), 2.30-2.15 (m, 2H), 2.05-1.78 (m, 2H), 1.60-1.46 (m, 2H), 1.39 (s, 3H), 1.31 (s, 3H), 1.21-1.05 (m, 2H). ¹³C NMR (75 MHz, d6-acetone) δ 178.9, 148.6, 144.4, 131.8, 129.9, 122.2, 119.8, 56.2, 44.5, 42.3, 38.0, 36.1, 34.0, 29.5, 21.4, 20.4, 16.8.

### Synthesis of Wu135 and Wu136

The mixture of podocarpic acid (PoCA) (40 mg, 0.146 mMol) and NCS (39.9 mg, 0.299 mMol) in 0.9 mL acetonitrile was irradiated under microwave at 100° C. for 1 h, conversion was not fully, another 8 mg of NCS was added and irradiated under microwave at 100° C. for another 30 min, then purified directly with preparative LC (20-90% acetonitrile in water, 10 mM NH₄OAc)) to give about 18.9 mg Wu134 (38% yield). ¹H NMR (300 MHz, d6-acetone) δ 7.07 (s, 1H), 3.46-3.35 (m, 1H), 2.83-2.74 (m, 2H), 2.30-2.15 (m, 2H), 2.05-1.78 (m, 2H), 1.60-1.46 (m, 2H), 1.39 (s, 3H), 1.31 (s, 3H), 1.21-1.05 (m, 2H). ¹³C NMR (75 MHz, d6-acetone) δ 178.9, 148.6, 144.4, 131.8, 129.9, 122.2, 119.8, 56.2, 44.5, 42.3, 38.0, 36.1, 34.0, 29.5, 21.4, 20.4, 16.8.

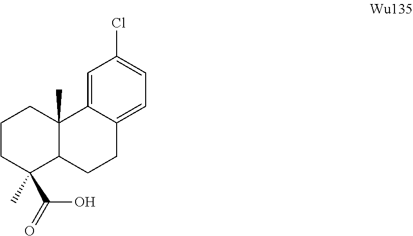 [SMILES: C[C@]1(C(=O)O)CCC[C@]2(C)c3cc(Cl)ccc3CCC12]

(1S,4aS)-6-chloro-1,4a-dimethyl-1,2,3,4,4a,9,10,10a-octahydrophenanthrene-1-carboxylic acid (Wu135) To Wu131 (50 mg, 0.162 mMol) in 4 mL DCM added pyridine (42.5 uL, 0.535 mMol) followed by triflate anhydride (76.7 uL, 0.453 mMol) at 0° C. and stirred at 0° C. for about 3 h. The reaction mixture was quenched with water, extracted with DCM (3 mL×2), filtered through the MgSO₄ and concentrated to give a crude product. Purified on preparative LC (35-90% acetonitrile in water, 10 mM NH₄OAc) to give about 25.5 mg product (36%).

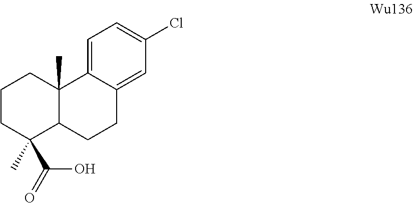 [SMILES: C[C@]1(C(=O)O)CCC[C@]2(C)c3ccc(Cl)cc3CCC12]

(1S,4aS)-7-chloro-1,4a-dimethyl-1,2,3,4,4a,9,10,10a-octahydrophenanthrene-1-carboxylic acid (Wu136)

### FIGURE LEGENDS

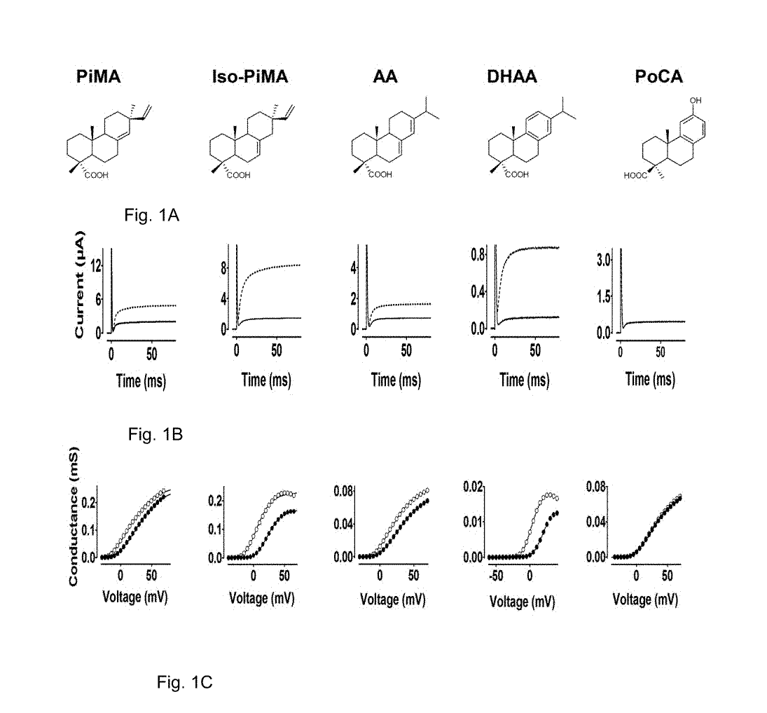

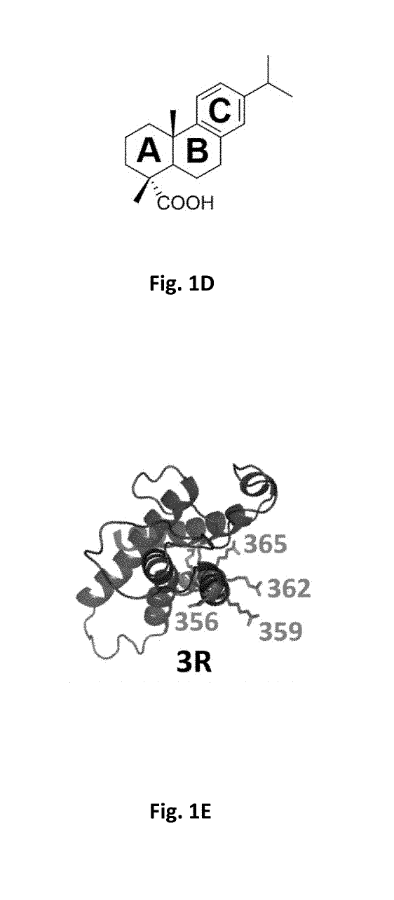

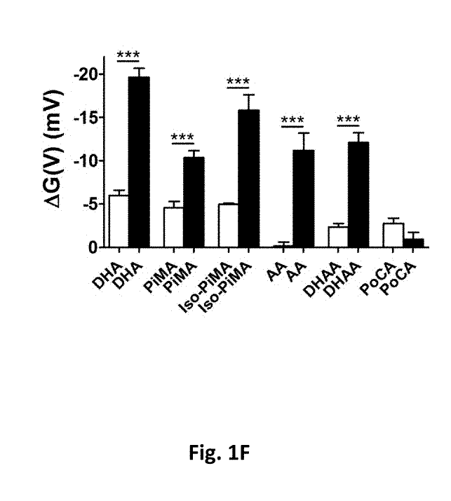

FIGS. 1a-f shows the effect of several natural resin acids on the opening of the Shaker K channel.

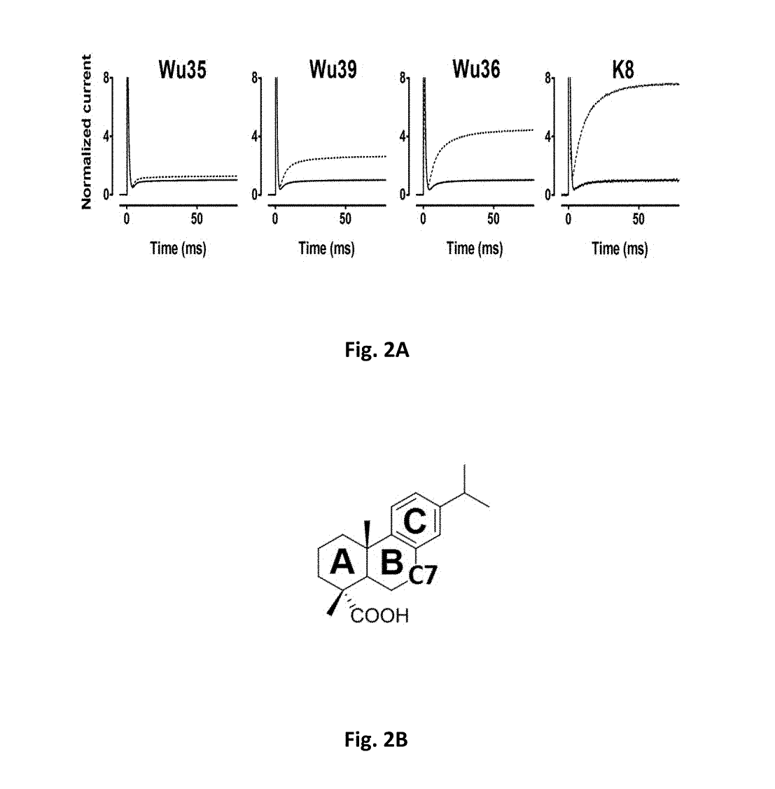

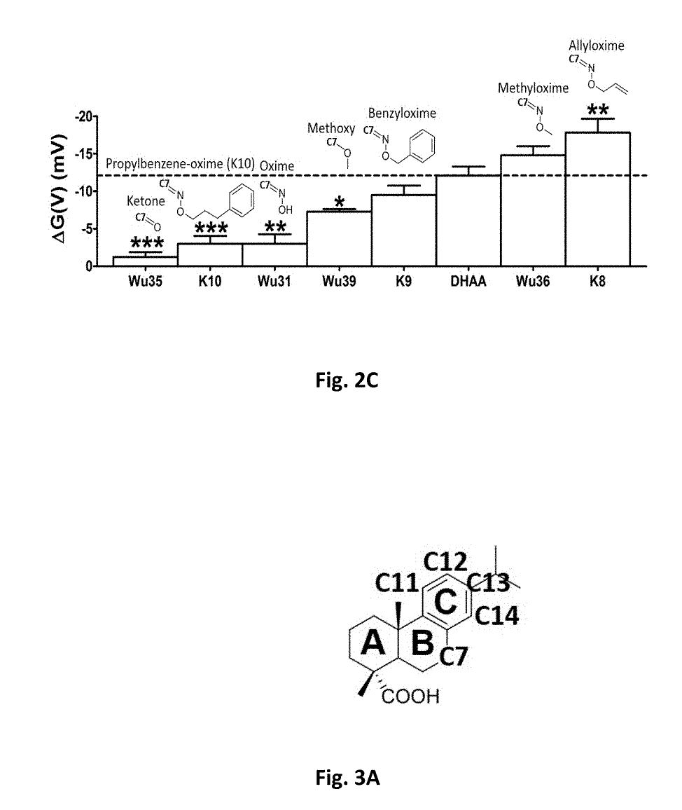

FIGS. 2a-c shows the efficacy of DHAA according to the invention modified at C7 at the B-ring to activate 3R Shaker K channel in comparison to DHAA.

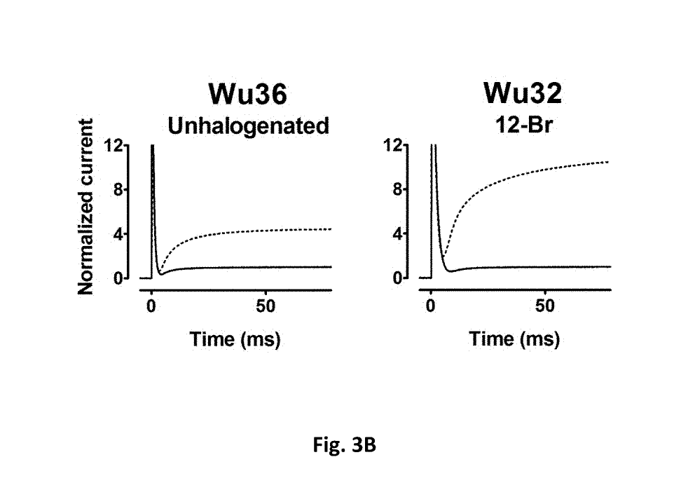

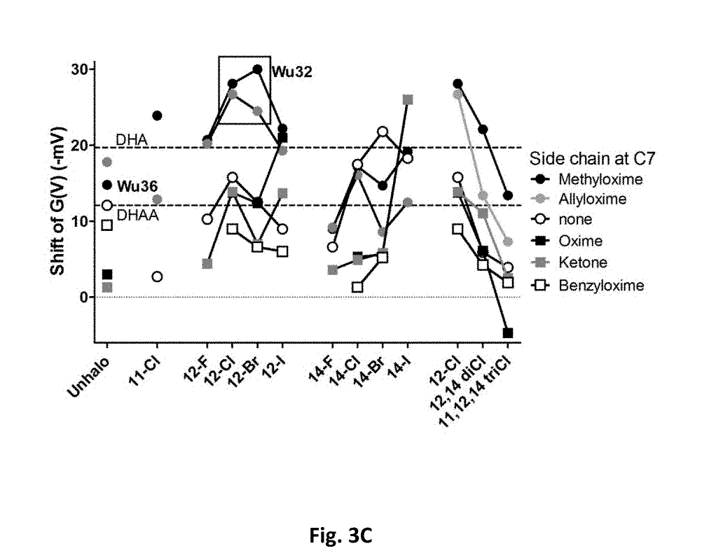

FIG. 3a-c shows potency variations for halogen modification of DHAA-derivatives according to the invention.

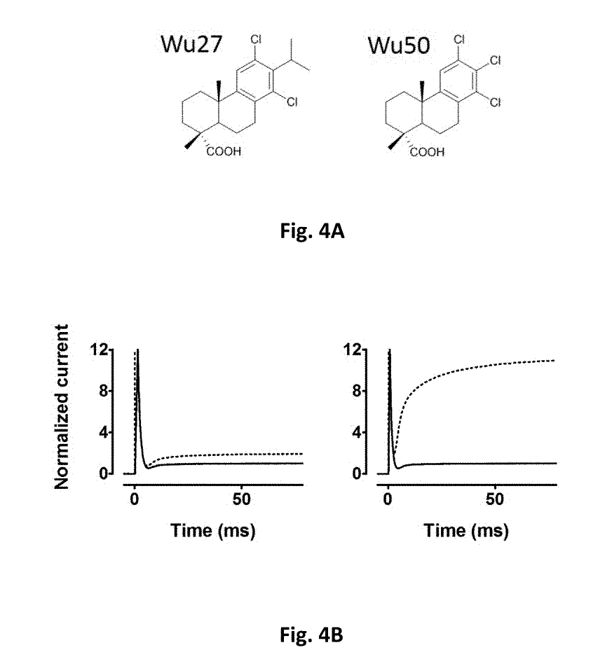

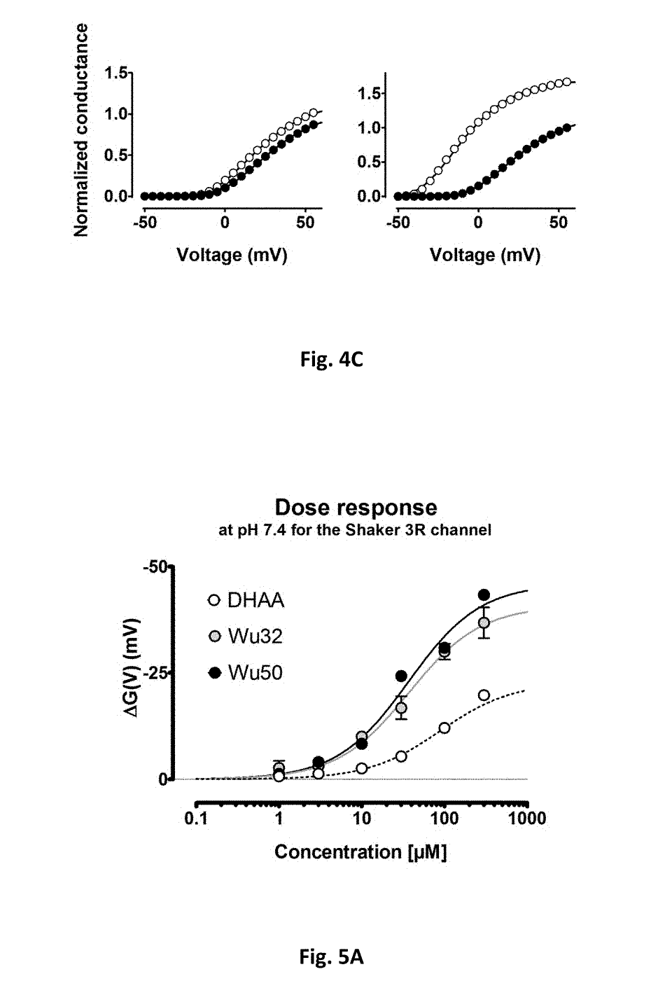

FIGS. 4a-c shows a comparison between DHAA derivatives according to the invention modified in C13.

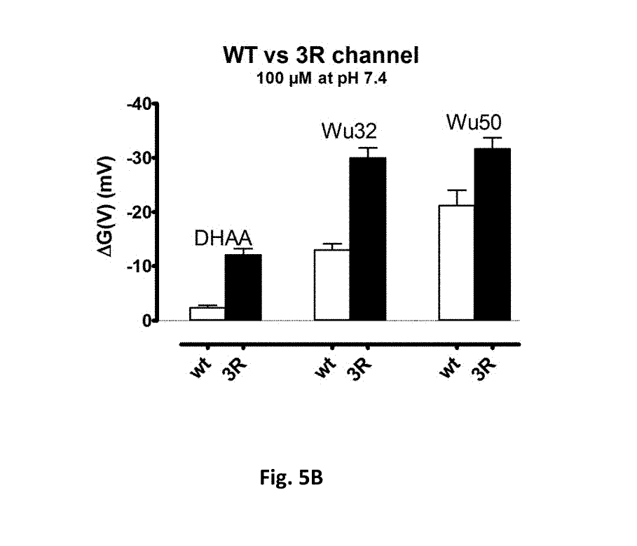

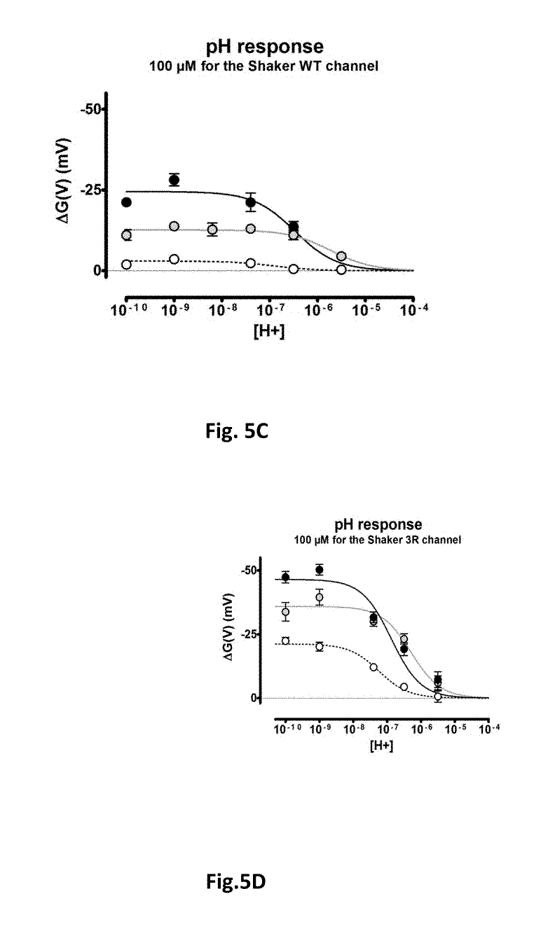

FIGS. 5a-d show dose and pH dependent compound sensitivity comparisons for DHAA and DHAA derivatives according to the invention.

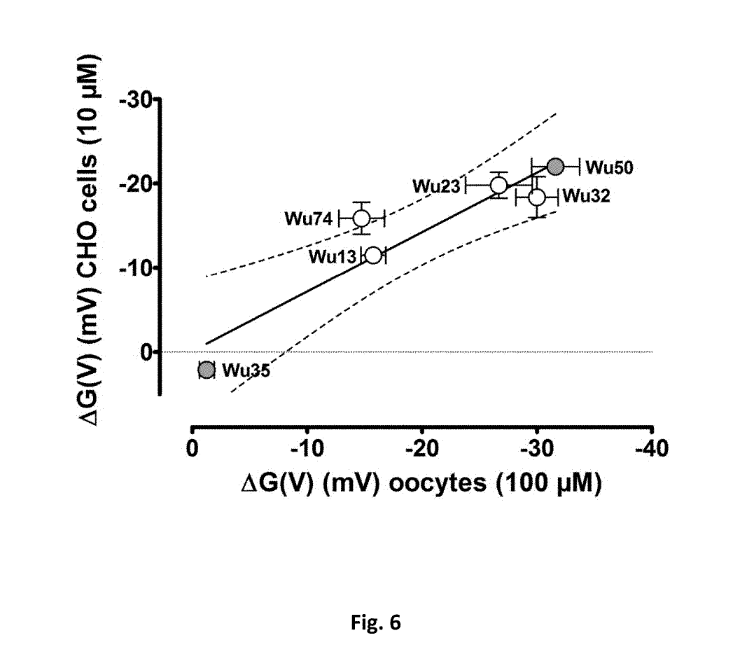

FIG. 6 shows correlations between the G(V) shifts for the 3R channel expressed in CHO-cells (10 μM at pH 7.4) versus Xenopus oocytes (100 μM at pH 7.4).

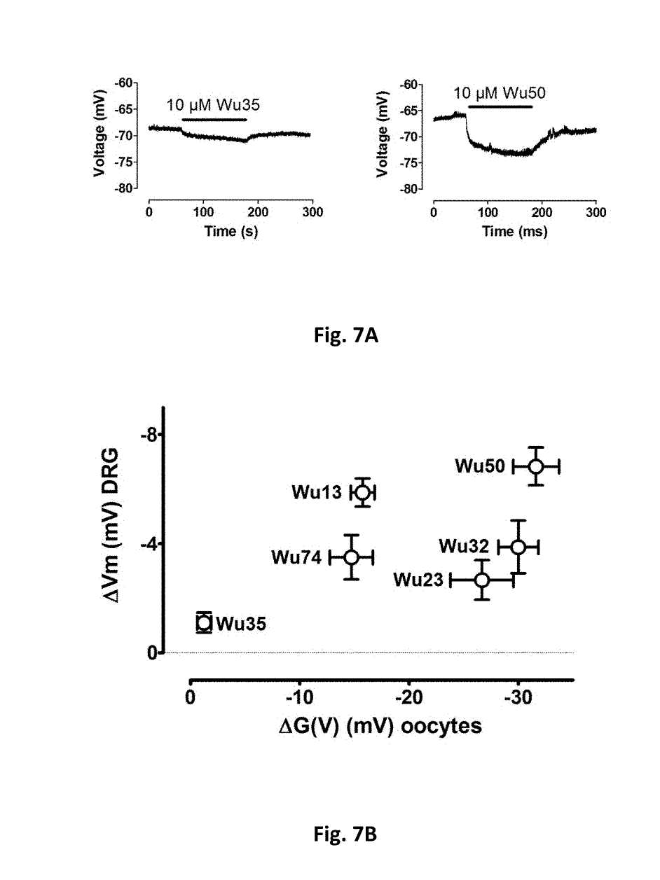

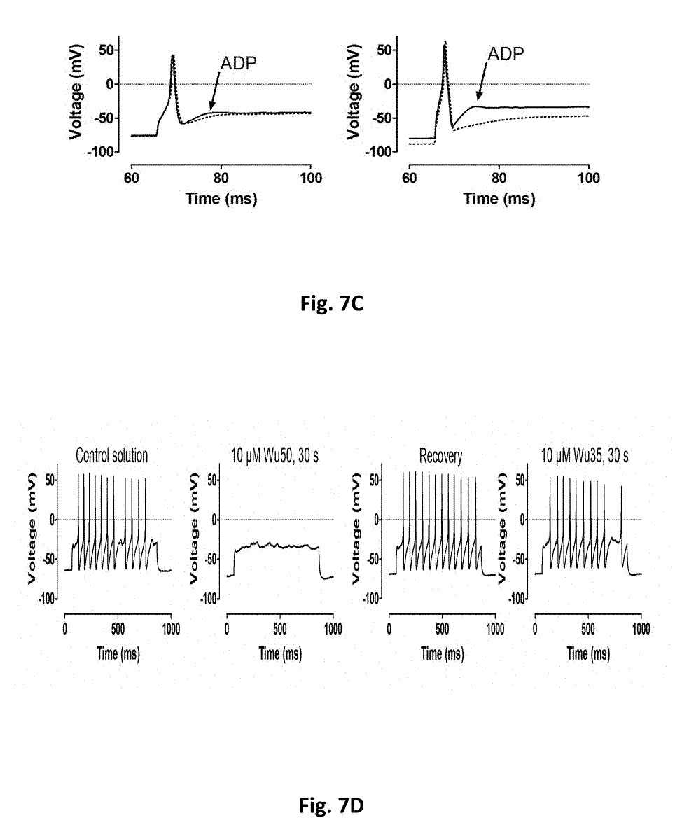

FIG. 7 shows the effect of DHAA derivatives on the resting potential and excitability of DRG neurons.

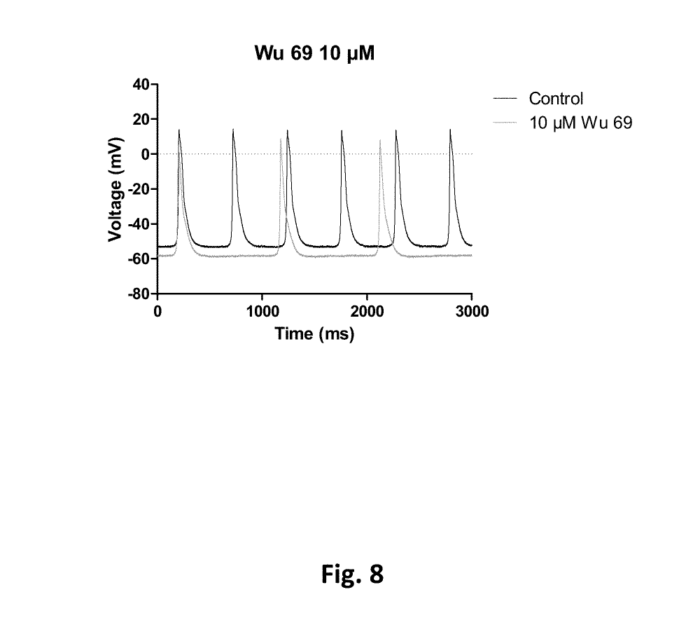

FIG. 8 shows the membrane potential of a spontaneously beating HL-1 cell. 10 uM of the (1R,4aS,E)-6-chloro-7-isopropyl-9-methoxyimino-1,4a-dimethyl-1,2,3,4,4a,9,10,10a-octahydrophenanthrene-1-carboxylic acid (Wu69) hyperpolarized the membrane potential and reduced the frequency.

### Example 1

### Natural Resin Acids Open the WT and the 3R Shaker K Channel

Five naturally occurring and commercially available resin acids were tested (FIG. 1a, pimaric acid, PiMA; isopimaric acid, iso-PiMA; abietic acid, AA; dehydroabietic acid, DHAA; and podocarpic acid, PoCA) at a concentration of 100 μM at pH 7.4 on the genetically modified 3R Shaker K channel (designed to be extrasensitive to electrically charged lipophilic, i.e. lipoelectric, compounds). The channel was expressed in oocytes from Xenopus laevis and currents were measured by the two-electrode voltage-clamp technique. FIG. 1a shows molecular structure for (left to right) pimaric acid (PiMA), isopimaric acid (Iso-PiMA), abietic acid (AA), dehydroabietic acid (DHAA), and podocarpic acid (PoCA). FIG. 1b shows representative current traces for voltages corresponding to 10% of maximum conductance in control solution at pH 7.4 of the 3R Shaker K channel. Black traces indicate control, and dotted traces 100 μM compound (same order as in a). The relative current amplitudes are by PiMA, 2.4 times; by Iso-PiMA, 5.7 times; by AA, 2.2 times; by DHAA, 4.6 times; by PoCA 1.0 times. FIG. 1c shows representative G(V) curves. Same cells as in FIG. 1b (control, black symbols; compound, non-filled symbols. ΔG(V) (left to right)=−10.8, −20.0, −8.1, −15.5, and +0.1 mV in these examples.

FIG. 1d shows the nomenclature for the naming of the carbon atoms in the compound skeleton. In FIGS. 1b and c it is shown that four of the five resin acids had clear effects of the channel's voltage sensitivity, but none was more potent than DHA, which has been used in previous studies on the lipoelectric mechanism. Moving the double bond from the C ring (PiMA) to the B ring (Iso-PiMA), see FIG. 1d, increased the shift of the conductance-versus-voltage, G(V), curve from −10.4±0.8 (n=6) to −15.9±1.8 mV (n=4; p=0.013). This double bond movement makes the B-ring less bulky and shifts the higher electron density connected to the double bond from the C ring to the B ring (FIG. 1c). In contrast, the structural difference between AA (conjugated double bond) and DHAA (aromatic C ring) does not generate any difference in potency between AA (−11.2±2 mV, n=5) and DHAA (−12.1±1.7 mV, n=6). In contrast to the other resin acids, PoCA, having a highly polar OH-group at the C-ring, had no effect on the 3R Shaker K channel (−1.4±1.1; n=5).

To explore if the investigated substances acted via a lipoelectric mechanism, resin acids were tested on the WT Shaker K channel (where 359 ad 356 are non-charged, FIG. 1e). In the 3R channel, 356, 359, 362 and 365 are arginines. Compound-induced G(V) shifts for the WT (gray) and 3R Shaker K channel (black) are shown in FIG. 1f (Mean±SEM; n=9, 15, 15, 6, 4, 4, 5, 5, 10, 6, 4, and 6 from left to right). The shifts of WT and 3R Shaker K channel are compared for each compound (one-way ANOVA together with Bonferroni's multiple comparison test: *, P<0.05; ***, P<0.0001). It is shown that DHAA, AA, PiMA and iso-PiMA had much smaller effects on WT compared to the 3R Shaker K channel, suggesting they all acted via the lipoelectric mechanism. However, AA did not show any effects at all on WT, suggesting that this compound probably will be difficult to turn into a potent compound on the WT channel. PoCA on the other hand had no effect on the 3R Shaker K channel and the effect on WT was not significantly different from the 3R Shaker K channel.

### Example 2

### Modification of the B-Ring of DHAA

Divergent substitutions were introduced on the B-ring of DHAA. We synthesized seven different side chains on C7 (from left to right: DHAA with C7 marked, Wu35, K10, Wu31, Wu39, K9, Wu36, and K8 (FIG. 2c). FIG. 2a shows representative current traces for voltages corresponding to 10% of maximum conductance in control solution at pH 7.4 of the 3R Shaker K channel. Black traces indicate control, and dotted traces 100 μM of the compound The relative current amplitudes are by Wu35, 1.3 times; by Wu39, 2.6 times; by Wu36, 4.4 times, and by K8, 7.6 times

FIG. 2b shows from left to right: molecular structure for DHAA with C7 marked; molecular structures for the introduced side chain at C7 of DHAA for the indicated compounds. FIG. 2c shows compound induced G(V) shifts for the 3R Shaker K channel. The dashed line equals the DHAA-induced shift. Mean±SEM (n (from left to right)=4, 5, 4, 4, 6, 4, and 4). The shifts are compared with DHAA (one-way ANOVA together with Dunnett's multiple comparison test: *, P<0.05; **, P<0.01; ***, P<0.001).

All polar substituents (Wu35, Wu31, and Wu39) clearly reduced the current increase by reducing the absolute G(V) shift. The polarity probably makes it more difficult for the compound to integrate in the membrane. However, the introduction of the non-polar propylbenze connected to the oxime group for K10 also caused a decreased potency compared to DHAA. This molecule may be too bulky, which might complicate the integration in the membrane in close proximity to the voltage sensor. In support of this, shortening the chain length (K9) restored the potency. Taken together, these compounds indicate that the chemical properties of the B-ring might be important for interaction with the lipid bilayer or else have some steric effect for the interaction between the compound and the ion channel. Two compounds had no significantly different effects compared to DHAA (Wu36 and K9). One compound (K8, an allyloxime on C7) significantly increased the effect (ΔG(V)=−17.8±1.9; n=4). It is demonstrated that an allyl at the oxime group causes an even higher potency than DHAA.

### Example 3

### Halogenated DHAA Derivatives

To increase the potency of the DHAA derivatives to open the 3R Shaker K channel, different halogens were introduced to C11, C12 and/or C14 in the C ring in combination with the different side chains at C7 (see FIG. 3a). Their potency to shift the voltage-dependence of activation of the 3R Shaker K channel was measured at 100 μM and pH 7.4 FIG. 3b shows representative current traces for voltages corresponding to 10% of maximum conductance in control solution at pH 7.4 of the 3R Shaker K channel. Black traces indicate control, and dotted traces 100 μM of the compound (from left to right: Wu36, Wu32). The relative current amplitudes are by Wu36, 4.4 times; and by Wu32, 10.8 times. FIG. 3c shows shift of G(V) (mean values) induced by the unhalogenated and halogenated DHAA derivatives. The upper dashed line is equal to the shift induced by 100 μM of DHA for the 3R Shaker K channel (n=4-9). The lower dashed line is equal to the shift induced by 100 uM DHAA. The symbols are coded according to the side chain at C7. The compounds presented in FIG. 3b are marked in FIG. 3b. FIG. 3b demonstrates that bromination of C12 with a methyloxime at C7 increases the G(V) shift by a factor of 2.5 The four most potent combinations contained either a chlorine or a bromine at C12 together with either a methyloxime or an allyloxime at C7 (box in FIG. 3c). The G(V) shifts were −24.5 to −30.0 mV compared to −12.1 mV for DHAA. In general these experiments demonstrate that, (i) halogenation of C12 increases the effect excepts if C7 lack a side chain or is very bulky (benzyloxime), (ii) halogenation of C14 has modest effects, (iii) fluorination is the least effective halogenation, (iv) double and triple chlorination reduces the effect compared to chlorination of C12 alone.

These tests indicate that the effects introduced by halogenation in the C ring and by a side chain at C7 may not be not additive. As an example, adding a methyloxime to C7 (Wu36) increased the potency from −12.1 to −14.8 mV (ΔV_{c7}=−2.7 mV), and adding a Br-12 to the C ring (Wu24) increased the potency from −12.1 to −12.4 (ΔVₕₐₗₒ=−0.3 mV). But adding a methyloxime to C7 in combination with Br-12 to the C ring (Wu32) increased the potency from −12.1 to −30.0; the overall increase in shift ΔVₜₒₜ=−18.5 mV, that is −15.5 mV more than expected from simple addition.

Thus, to summarize, neither halogenation of C12, nor a side chain at C7 had a large effect per se, but a combination of smaller hydrophobic (methyloxime and allyloxime) side chains at C7 and halogenation at C12 had a large effect. In contrast, halogenation (Cl, Br or I) of C14 had a slightly larger effect per se, but in combination with the side chains at C7 the effect was largely suppressed.

### Example 4

### DHAA Derivatives with Modifications in Position C13

FIG. 4a shows the molecular structure for the DHAA derivatives Wu27 (left) and Wu50 (right). Wu50, similar to Wu27 but with the isopropyl of the C-ring replaced by a chloride were synthesized and tested on the 3R Shaker K channel. FIG. 4b shows representative current traces for voltages corresponding to 10% of maximum conductance in control solution at pH 7.4 of the 3R Shaker K channel. Black traces indicate control, and dotted traces 100 μM compound (left, Wu27; right, Wu50). The relative current amplitudes are by Wu27, 1.9 times; by Wu50, 11.1 times. FIG. 4c shows representative G(V) curves. Same cells as in FIG. 4b (control, black symbols; compound, non-filled symbols. AG(V)=−6.1 mV by Wu27, and −32.6 mV by Wu50 in these examples. The potency of Wu50 was, in contrast to Wu27, very high. For the 3R Shaker K channel, 100 μM Wu50 at pH 7.4 caused more than a ten-fold increase of the current compared to less than a twofold increase for Wu27 (FIG. 4b) and also a five times larger shift of the voltage dependence of activation by −31.6±2.1 (n=9) compared to −5.9±0.4 (n=5) for Wu50 and Wu27 respectively (FIG. 4c).

### Example 5

### Increased Channel-Opening Propensity Depends on Decreased pKₐ Value, Increased Affinity, and Increased Effect of the Deprotonated Compound

The present experiments have identified ten compounds more potent than DHA and 32 compounds more potent than DHAA. To investigate if the derivatives acts similar to DHA and to explore if the difference in potencies among the derivatives depends on altered pKₐ values, on altered affinities, or on altered maximum effects the effects were tested at (i) different concentrations, at (ii) different pH, and on (iii) two different channels (WT vs. 3R). The mother compound (DHAA) and the two most potent derivatives (Wu32, and Wu50) were tested.

FIG. 5a shows a dose dependence curve for DHAA (non-filled), Wu32 (shaded), and Wu50 (black) at pH 7.4 for the 3R Shaker K channel. Error bars indicate SEM (n=4-9). FIG. 5b shows how the compounds induced G(V) shifts for the WT and 3R Shaker K channels. FIGS. 5c-d show pH dependence curves for WT (c) and 3R Shaker K channel (d) for 100 μM DHAA (non-filled), 100 μM Wu32 (shaded), and 100 μM Wu50 (black). Error bars indicate SEM (n=2-9).

The tests demonstrate that Wu50 was most potent and induced a significant shift already at 1 μM and pH 7.4 on the 3R Shaker K channel (ΔG(V)−1.3±0.3 (n=9)) (see FIG. 5a). The K_{d}-value was 89 μM for DHAA, and 37 μM for both derivatives. The maximum shift was −23 mV for DHAA, and −41 and −46 for Wu32 and Wu50 respectively. Thus, both the affinity and the maximum effect were increased. Another way to describe the effect is to determine the concentration that shifts the G(V) by −10 mV; 10-12 μM of Wu50 or Wu32 is required while 70 μM DHAA is required for the same effect. For all three compounds, the induced shifts are smaller on Shaker WT compared to Shaker 3R (see FIG. 5b), thus supporting a similar mechanism. Notably, 100 μM Wu50 at pH 7.4 shifts the G(V) for WT almost 10 times more compared 100 μM DHAA at pH 7.4, −21.2 mV vs. −2.3 mV.

The pKₐ value for DHAA was 7.3 and 7.2 for the 3R and WT channels respectively (FIG. 5c-d). The pKₐ values for Wu32 are 5.8 and 6.1 respectively, and the pKₐ values for Wu50 are 6.5 and 6.8 respectively (FIG. 5c-d), and thus the derivatives but not the mother substance DHAA are almost fully deprotonated at neutral pH. Taken together, modifications increasing the effect had profound effect on the pKₐ value (up to 1.5 pH steps), the affinity and the maximum effect.

### Example 6

### DHAA Derivatives are More Potent in a Mammalian Expression System

The Xenopus oocyte an expression system can affect the absolute concentration required to reach a certain effect compared to mammalian cells. To test if the effects reported on K channels expressed in Xenopus oocytes in the present investigation also apply to K channels expressed in mammalian cells we investigated the effects of selected compounds on a Chinese hamster ovary (CHO) cell line stably expressing the 3R Shaker K channel by conventional whole-cell patch-clamp recordings. FIG. 6 demonstrates that shifts induced by 10 μM resin-acid derivatives in the CHO cell correlated well with shifts induced by 100 μM in the Xenopus oocyte. It can be concluded that the found G(V) shifting property of the compounds is a general effect coupled to the specific ion channel and not to the expression system.

Accordingly, the compounds synthesized in the context of the present invention act on K channels expressed in a mammalian cell line, and nine of the substances significantly open the channel at 3.3 μM. These substances have suitable characteristics to be developed into excitability-reducing substances. In the following example, these compounds are tested for their capacity to reduce excitability in a neuron with endogenously expressed channels.

### Example 7

### Resin-Acid Derivatives Hyperpolarized Dorsal Root Ganglion (DRG) Neurons and Reduced Neuronal Excitability

FIG. 7a shows recordings of Vm during application of the compound Wu13. FIG. 7b shows compound-induced hyperpolarizing shifts of Vm of DRG neurons (10 μM at pH 7.4) plotted versus the shifts for the 3R Shaker K channel expressed in Xenopus oocytes (100 μM at pH 7.4).

The compounds tested on the 3R Shaker K channel expressing CHO cells with the patch-clamp technique were also tested on native dorsal root ganglion (DRG) neurons from mice. All of the tested compounds caused a shift of the resting membrane potential (ΔVₘ) towards more negative voltages (p<0.05; FIG. 7a,b). This hyperpolarization most likely depends on the opening of one or several K channels. The excitability of the neurons is expected to be reduced by changing the threshold for the input to evoke an action potential. Wu35, the least potent G(V) shifter of the investigated compounds, caused only a small shift of the resting potential (ΔVₘ=−1.1±0.4; n=5). Wu50, the most potent shifter, caused the largest hyperpolarization (ΔVₘ=−6.8±0.7; n=6). Even though there was not a linear relation between G(V) shift and the hyperpolarization, the trend is clear (see FIG. 7b). One obvious reason for this deviation is that the channel composition is different in DRG neurons from 3R Shaker K channel expressing CHO cells or Xenopus oocytes. The two most potent compounds on the DRG neurons (Wu13 and Wu50) have in common to lack modifications of the B-ring and to have a chlorine at C12. The intermediately potent hyperpolarizers (Wu23, 32, 74) have side chains on C7. Thus a side chain on C7 may be less important for hyperpolarizations.

To test if the compounds also reduced excitability, we stimulated the DRG neurons by a constant current pulse. In a cell where only a single action potential was elicited, 10 μM Wu35 had almost no effect the resting potential and only a very small effect on the after hyperpolarization (FIG. 7c left). In contrast, 10 μM Wu50 made the resting potential more negative and had a pronounced effect on the after hyperpolarization, indicating that a K channel stayed open a long time after the action potential with the potential to reduce excitability (FIG. 7c right). In another type of neuron, a continuous pulse generated a train of action potentials (FIG. 7d panel 1). 10 μM Wu50 completely abolished all action potentials (panel 2). The effect was clearly reversible (panel 3). In contrast, 10 μM Wu35, which was a very poor shifter in both the Xenopus oocytes and in the CHO cells, and which only had a minor effect on the resting potential, had almost no effect on the same cell (panel 4). Thus, the potent shifters are expected to reduce excitability.

### Summary of Examples 1-7

The present invention relates to the design, synthesis and functional characterization of several DHAA derivatives and their capacity as Kv ion openers and thereby their usefulness for treatment of hyperexcitability diseases. The invention includes demonstrating the usefulness of certain DHAA derivatives halogenated in position C12 and with certain hydrophobic side chains on C7 have increased the potency to open a K channel expressed in two different cellular systems. The channel-opening properties of the compounds were also correlated with the property to reduce excitability in a DRG neuron. Thus, several of the described resin-acid derivatives have the potential to be developed into medical drugs to reduce neuronal excitability.

For example, the DHAA derivative termed Wu50, wherein the isopropyl group at C12 is changed to a chlorine and wherein chlorines are added at C11 and C13 leads to a molecule leads to a potent molecule which, shifts the G(V) of WT at 100 μM and pH 7.4 by −21.2 mV, compared to only −2.3 mV for the mother substance DHAA, and −6.0 for the previously most potent lipoelectric compound DHA. At relatively negative voltages, critical for repetitive firing in neurons, these shifts can be converted to increases in current amplitude, A=exp(−ΔV/4.7). The amplitude increase for Wu50 compared DHA is thus A_{wu50}/A_{DHA}=exp(−ΔV/4.7, which gives a factor of 25. Thus, Wu50 makes the current 25 times bigger than DHA. Altering DHAA by changing the isopropyl group at C12 to a chlorine and adding chlorines at C11 and C13 leads to a molecule (Wu50) that makes the current 56 times bigger of the WT channel at pH 7.4.

Some of the compounds described herein have been reported to affect the current through large-conductance Ca-activated K (i.e. BK) channels, see Y-M Cui et al. Bioorg Med Chem, 18, 8642-8659. However, the BK channel may contain different binding sites than the Kv ion channels and diverse effects have been found on the BK channel compared to the 3R channel. For this reason, compounds demonstrating potency for the BK channels cannot be expected to be highly potent for the Kv channel and vice versa.

Table 1 below shows ΔG(V) (mV) values according to the tests of Examples 1 to 5 for different DHAA derivatives. The derivatives can be substituted in carbons 7, 11, 12, 13 and 14 of the DHAA ring system. In Table 1 DHAA denotes dehydroabietic acid PoCA denotes podocarpic acid. The compounds of Table 1 can be useful for treatment of cardiac arrhythmia, or a hyperexcitability disease, such as pain or epilepsy when administered in a therapeutically acceptable dose.

TABLE 1

| | | | | | | | G(V) shift |
|---|---|---|---|---|---|---|---|
| Substance | Template | C7 | C11 | C12 | C13 | C14 | (mV) |
| DHAA | DHAA | — | — | — | isopropyl | — | −12.1 |
| K8 | DHAA | Allyloxime | — | — | isopropyl | — | −17.8 |
| K9 | DHAA | Benzyloxime | — | — | isopropyl | — | −9.5 |
| K10 | DHAA | Propylbenzene- | — | — | isopropyl | — | −3.0 |
| | | oxime | | | | | |
| Wu13 | DHAA | — | — | Cl | Isopropyl | — | −15.8 |
| Wu14 | DHAA | — | — | — | Isopropyl | Cl | −17.5 |
| Wu15 | DHAA | carbonyl | — | — | Isopropyl | Cl | −4.9 |
| Wu16 | DHAA | Methyloxime | — | — | Isopropyl | Cl | −17.2 |
| Wu17 | DHAA | Oxime | — | — | Isopropyl | Cl | −5.3 |
| Wu18 | DHAA | Benzyloxime | — | — | Isopropyl | Cl | −1.3 |
| Wu19 | DHAA | Allyloxime | — | — | Isopropyl | Cl | −16.1 |
| Wu20 | DHAA | carbonyl | — | Cl | Isopropyl | — | −13.9 |
| Wu21 | DHAA | Oxime | — | Cl | Isopropyl | — | −13.8 |
| Wu22 | DHAA | Benzyloxime | — | Cl | Isopropyl | — | −9.1 |
| Wu23 | DHAA | Allyloxime | — | Cl | Isopropyl | — | −26.7 |
| Wu24 | DHAA | — | — | Br | Isopropyl | — | −12.5 |
| Wu26 | DHAA | — | — | — | Isopropyl | Br | −21.8 |
| Wu27 | DHAA | — | — | Cl | Isopropyl | Cl | −5.9 |
| Wu28 | DHAA | Oxime | — | Br | Isopropyl | — | −12.4 |
| Wu30 | DHAA | carbonyl | — | Br | Isopropyl | — | −7.0 |
| Wu31 | DHAA | Oxime | — | — | Isopropyl | — | −3.0 |
| Wu32 | DHAA | Methyloxime | — | Br | Isopropyl | — | −30.0 |
| Wu33 | DHAA | Allyloxime | — | Br | Isopropyl | — | −24.5 |
| Wu34 | DHAA | Benzyloxime | — | Br | Isopropyl | — | −6.6 |
| Wu35 | DHAA | carbonyl | — | — | Isopropyl | — | −1.3 |
| Wu36 | DHAA | Methyloxime | — | — | Isopropyl | — | −14.8 |
| Wu37 | DHAA | carbonyl | — | Cl | Isopropyl | Cl | −11.1 |
| Wu39 | DHAA | Methoxy | — | — | Isopropyl | — | −7.3 |
| Wu40 | DHAA | Allyloxime | — | Cl | Isopropyl | Cl | −13.4 |
| Wu41 | DHAA | Methyloxime | — | Cl | Isopropyl | Cl | −22.1 |
| Wu42 | DHAA | Oxime | — | Cl | Isopropyl | Cl | −6.1 |
| Wu43 | DHAA | Benzyloxime | — | Cl | Isopropyl | Cl | −4.2 |
| Wu45 | DHAA | — | Cl | Cl | Isopropyl | Cl | −3.9 |
| Wu46 | DHAA | carbonyl | Cl | Cl | Isopropyl | Cl | −2.5 |
| Wu47 | DHAA | Methyloxime | Cl | Cl | Isopropyl | Cl | −13.4 |
| Wu48 | DHAA | Oxime | Cl | Cl | Isopropyl | Cl | 4.7 |
| Wu49 | DHAA | Allyloxime | Cl | Cl | Isopropyl | Cl | −7.3 |
| Wu50 | DHAA | — | — | Cl | Cl | Cl | −31.6 |
| Wu51 | DHAA | Benzyloxime | Cl | Cl | Isopropyl | Cl | −1.9 |
| Wu52 | DHAA | Allyloxime | — | F | Isopropyl | — | −20.2 |
| Wu53 | DHAA | Allyloxime | — | — | Isopropyl | F | −9.2 |
| Wu54 | DHAA | Methyloxime | — | — | Isopropyl | F | −9.0 |
| Wu55 | DHAA | Methyloxime | — | F | Isopropyl | — | −19.9 |
| Wu56 | DHAA | — | — | — | Isopropyl | F | −6.6 |
| Wu60 | DHAA | — | — | I | Isopropyl | — | −9.0 |
| Wu61 | DHAA | — | — | — | Isopropyl | I | −18.3 |
| Wu62 | DHAA | carbonyl | — | I | isopropyl | — | −13.7 |
| Wu63 | DHAA | Allyloxime | — | I | Isopropyl | — | −19.3 |
| Wu64 | DHAA | Methyloxime | — | I | Isopropyl | — | −22.2 |
| Wu68 | DHAA | Carbonyl | — | — | Isopropyl | I | −26.0 |
| Wu69 | DHAA | Methyloxime | — | Cl | Isopropyl | — | −28.1 |
| Wu73 | DHAA | carbonyl | — | — | Isopropyl | Br | −5.8 |
| Wu74 | DHAA | Methyloxime | — | — | Isopropyl | Br | −14.7 |
| Wu75 | DHAA | Allyloxime | — | — | Isopropyl | Br | −8.6 |
| Wu76 | DHAA | — | — | — | Isopropyl | Vinyl | −7.7 |
| Wu78 | DHAA | — | — | Vinyl | Isopropyl | — | −13.2 |
| Wu81 | DHAA | — | — | cyclopropyl | Isopropyl | — | −8.2 |
| Wu84 | DHAA | — | — | Propenyl | Isopropyl | — | −0.2 |
| Wu85 | DHAA | — | — | — | Isopropyl | Propenyl | −1.9 |
| Wu86 | DHAA | Oxime | — | I | Isopropyl | — | −21.0 |
| Wu87 | DHAA | Benzyloxime | — | I | Isopropyl | — | −6.0 |
| Wu88 | DHAA | Oxime | — | — | Isopropyl | Br | −5.6 |
| Wu89 | DHAA | Benzyloxime | — | — | Isopropyl | Br | −5.2 |
| Wu90 | DHAA | Methyloxime | — | — | Isopropyl | I | −19.2 |
| Wu91 | DHAA | Allyloxime | — | — | Isopropyl | I | −12.5 |
| Wu100 | DHAA | — | Cl | — | Isopropyl | — | −2.7 |
| Wu101 | DHAA | Hydroxyl | Cl | — | Isopropyl | — | −9.6 |
| Wu102 | DHAA | Hydroxyl | Cl | — | Isopropyl | — | −2.8 |
| Wu104 | DHAA | Allyloxime | Cl | — | Isoprpyl | — | −12.9 |
| Wu105 | DHAA | Methyloxime | Cl | — | isopropyl | — | −23.2 |
| Wu106 | DHAA | — | Cl | — | Isopropyl | Hydroxyl | −3.4 |
| Wu108 | DHAA | — | — | F | Isopropyl | — | −10.4 |
| Wu112 | DHAA | — | Cl | — | isopropyl | Cl | −14.7 |
| Wu113 | DHAA | carbonyl | — | — | isopropyl | F | −3.6 |
| Wu114 | DHAA | Allyloxime | Cl | — | isopropyl | Cl | −13.3 |
| Wu115 | DHAA | — | — | — | Cl | Cl | −24.2 |
| Wu116 | DHAA | carbonyl | — | F | isopropyl | — | −4.4 |
| Wu118 | DHAA | carbonyl | — | vinyl | isopropyl | — | 0.2 |
| Wu119 | DHAA | Methyloxime | — | vinyl | isopropyl | — | −30.3 |
| Wu120 | DHAA | Allyloxime | — | vinyl | isopropyl | — | −26.1 |
| Wu121 | DHAA | carbonyl | — | cyclopropyl | isopropyl | — | −6.7 |
| Wu122 | DHAA | Methyloxime | — | cyclopropyl | isopropyl | — | −32.3 |
| Wu123 | DHAA | Allyloxime | — | cyclopropyl | isopropyl | — | −26.3 |
| Wu124 | DHAA | — | — | — | isopropyl | Phenyl | −0.4 |
| Wu127 | DHAA | — | — | — | isopropyl | Cyclopropyl | −9.6 |
| Wu129 | PoCA | — | — | — | — | — | −5.1 |
| Wu133 | DHAA | — | Cl | Cl | Cl | Cl | −25.6 |
| Wu135 | PoCA | — | — | Cl | — | — | −12.0 |
| Wu136 | PoCA | — | — | — | Cl | — | −17.8 |

### Example 8

### Effects of DHAA Derivatives in Cardiac Excitability

### Materials

All conventional chemicals were purchased from Sigma-Aldrich (Stockholm, Sweden). The DHAA derivatives were diluted in ethanol in a 100 mM stock solution. The stock solutions were stored at −20 t for further use.

### Cell Culture

The cell culture was done according to a previous study (Claycomb et al., Proceedings of the National Academy of Sciences of the United States of America 1998; 95:2979-2984). In brief, cells were grown on gelating fibronectin coated T75 flasks. They were maintained in Claycomb Medium which was supplemented with 10% fetal bovine serum, 2 mM L-glutamine, 0.1 mM noradrenaline, and 100 U/ml, 100 μg/ml penicillin-streptomycin. Enzymatic dissociation with 0.05% trypsin-EDTA was done after full confluence. Isolated cells were plated on fibronectin-gelatin coated plastic coverslips and used for recording.

### Electrophysiology

Whole-cell current-clamp recordings were done on confluent cells at 35±1° C., using an Axopatch 200B amplifier (Molecular Devices, Sunnyvale, Calif.). Patch pipettes were fabricated from borosilicate capillary glass on a vertical pipette puller and had resistances of 3-5 MO. Data were stored on a computer through Digidata 1440A interface (Molecular Devices), and was analyzed with pCLAMP software (version 10.1, Molecular Devices). The bath solution contained the following (in mM): 140 NaCl, 5.4 KCl, 1 MgCl₂, 1.8 CaCl₂, 10 HEPES, and 10 glucose (pH adjusted to 7.4 with NaOH). The micropipettes were filled with a solution containing (in mM): 130 K-gluconate, 9 KCl, 8 NaCl, 1 MgCl₂, 10 EGTA, 10 HEPES, and 3 Na₂ATP (pH adjusted to 7.3 with KOH).

### DHAA-Derivatives

The following compounds were used in the tests:
(1R,4aS,E)-9-((allyloxy)imino-7-isopropyl-1,4a-dimethyl-1,2,3,4,4a,9,10,10a-octahydrophenanthrene-1-carboxylic acid;
(1R,4aS)-8-bromo-7-isopropyl-1,4a-dimethyl-1,2,3,4,4a,9,10,10a-octahydrophenanthrene-1-carboxylic acid;
1R,4aS)-6-bromo-7-isopropyl-9-methoxyimino-1,4a-dimethyl-1,2,3,4,4a,9,10,10a-octahydrophenanthrene-1-carboxylic acid;
1R,4aS)-7-isopropyl-9-carboxy-1,4a-dimethyl-1,2,3,4,4a,9,10,10a-octahydrophenanthrene-1-carboxylic acid; and
(1R,4aS,E)-6-chloro-7-isopropyl-9-methoxyimino-1,4a-dimethyl-1,2,3,4,4a,9,10,10a-octahydrophenanthrene-1-carboxylic acid; and
(1R,4aS)-6,7,8-trichloro-1,4a-dimethyl-1,2,3,4,4a,9,10,10a-octahydrophenanthrene-1-carboxylic acid.

### Results

Whole-cell current-clamp recordings were done on spontaneously firing HL-1 cells, before and after the application of 10 uM of the DHAA derivatives. A typical recording is shown in FIG. 8 for the DHAA derivative (1R,4aS,E)-6-chloro-7-isopropyl-9-methoxyimino-1,4a-dimethyl-1,2,3,4,4a,9,10,10a-octahydrophenanthrene-1-carboxylic acid (Wu69). The interspike membrane potential is made more negative by 10 uM Wu69 and the frequency is reduced. A reduced frequency is what is expected from an antiarrhythmic drug.

Several tested DHAA derivative both hyperpolarized the membrane potential and reduced the frequency. In general, if the substances had a large effect on the Xenopus oocyte, they also hyperpolarized the cardiomyocytes and reduced the frequency. The quantify the effects we listed the effects on the membrane potential for the tested substances in Table 2 together with the effects on the K channel expressed in Xenopus oocytes.

TABLE 2

| | | | | | | Effects | |
|---|---|---|---|---|---|---|---|
| | | | | | | on K channel | on HL-1 |
| | | | | | | Mean ± SEM | cells Mean |
| Name | C7 | C11 | C12 | C13 | C14 | (mV) | (mV) |
| K8 | Allyloxime | — | — | isopropyl | — | −17.8 ± 1.9 | −3.6 |
| Wu26 | — | — | — | Isopropyl | Br | −21.8 ± 0.8 | −4.7 |
| Wu32 | Methyloxime | — | Br | Isopropyl | — | −30.0 ± 1.8 | −3.6 |
| Wu35 | carbonyl | — | — | Isopropyl | — | −1.2 ± 0.6 | 1.5 |
| Wu50 | — | — | Cl | Cl | Cl | −31.6 ± 2.1 | −5.5 |
| Wu69 | Methyloxime | — | I | Isopropyl | — | −28.1 ± 1.7 | −9.3 |

Table 2 demonstrates the effects of DHAA derivatives on the membrane potential of HL-1 cells.

The most potent substance on the HL-1 cells (Wu69) is also one of the most potent substances on the Shaker K 3R channel expressed in Xenopus oocytes. Wu35 which has no effect on the K channel has no effect on the HL-1 cells and a substance with intermediate effect on the K channel has an intermediate effect on the HL-1 cells. This suggest that if a DHAA derivative has an effect on the K channel it is also likely to have an effect, potentially antiarrhythmic effect, on HL-1 cells. However, there is no strict correlation between the effects on the two experimental systems: Wu32 which is very potent on the K channel only has an intermediate effect on the HL-1 cells.

We claim:

1. Dehydroabietic acid derivatives according to formula I and all stereoisomers thereof, wherein R₁₁, R₁₂, and R₁₄ are independently selected from hydrogen, halogen and R₂; R₁₃ is selected from hydrogen, halogen and R₃; and R₇ is selected from hydrogen, halogen, hydroxyl, carbonyl, and ═N—O—R₁; where R₁ is selected from hydrogen, and saturated or unsaturated lower alkyl groups selected from C1-C6 alkyl and C2-C6 alkenyl groups; R₂ and R₃ are independently from each other selected from straight, branched or cyclic saturated or unsaturated hydrocarbons comprising from 1 to 6 carbon atoms; for use in treatment of epilepsy, by extracellularly acting on the voltage sensor domain (VSD) to open at least one member of the family of voltage-gated Kv (potassium) channels (Kv family), wherein formula I is:

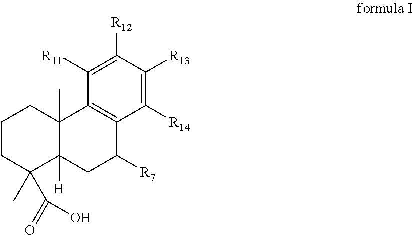

2. Dehydroabietic acid derivatives for use according to claim 1, wherein R₃ is isopropyl, selected from compounds included in the groups a) to m):
a) dehydroabietic acid derivatives according to formula I and all stereoisomers thereof, wherein R₁₂ is —F, R₁₁ and R₁₄ is —H, and R₇ is selected from —H, ═O and ═N—O—R₁, where R₁ is selected from —H, —CH₃, and —CH₂—CH═CH₂;
b) dehydroabietic acid derivatives according to formula I and all stereoisomers thereof, wherein R₁₁ and R₁₂ is —H, R₁₄ is —F, and R₇ is selected from —H, ═O and ═N—O—R₁, where R₁ is selected from —H, —CH₃, and —CH₂—CH═CH₂;
c) dehydroabietic acid derivatives, according to formula I and all stereoisomers thereof wherein R₁₂ is —Cl, R₁₁ and R₁₄ is —H, and R₇ is selected from —H, ═O and ═N—O—R₁, where R₁ is selected from —H, —CH₃, and —CH₂—CH═CH₂;
d) dehydroabietic acid derivatives according to formula I and all stereoisomers thereof, wherein R₁₁ and R₁₂ is —H, R₁₄ is —Cl, and R₇ is selected from —H, ═O and ═N—O—R₁, where R₁ is selected from —H, —CH₃, and —CH₂—CH═CH₂;
e) dehydroabietic acid derivatives according to formula I and all stereoisomers thereof, wherein R₁₁ is —Cl, R₁₂ and R₁₄ are —H, and R₇ and R₇ is selected from —H, ═O and ═N—O—R₁, where R₁ is selected from —H, —CH₃, and —CH₂—CH═CH₂;
f) dehydroabietic acid derivatives according to formula I and all stereoisomers thereof, wherein R₁₁ and R₁₄ are ═Cl, R₁₂ is —H, and R₇ is selected from —H, ═O and ═N—O—R₁, where R₁ is selected from —H, —CH₃, and —CH₂—CH═CH₂;
g) dehydroabietic acid derivatives, according to formula I and all stereoisomers thereof wherein R₁₁ and R₁₄ is —H, R₁₂ is —Br, and R₇ is selected from —H, ═O, ═N—O—R₁, where R₁ is selected from —H, —CH₃, and —CH₂—CH═CH₂;
h) dehydroabietic acid derivatives according to formula I and all stereoisomers thereof, wherein R₁₁ and R₁₂ is —H, R₁₄ is —Br, and R₇ is selected from —H, ═O and ═N—O—R₁, where R₁ is selected from —H, —CH₃, and —CH₂—CH═CH₂;
i) dehydroabietic acid derivatives according to formula I and all stereoisomers thereof, wherein R₁₂ is —I, R₁₁ and R₁₄ is —H, and R₇ is selected from —H, ═O and ═N—O—R₁, where R₁ is selected from —H, —CH₃, and —CH₂—CH═CH₂;
j) dehydroabietic acid derivatives according to formula I and all stereoisomers thereof, wherein R₁₁ and R₁₂ is —H, R₁₄ is —I, and R₇ is selected from —H, ═O and ═N—O—R₁, where R₁ is selected from —H, —CH₃, and —CH₂—CH═CH₂;
k) dehydroabietic acid derivatives according to formula I and all stereoisomers thereof, wherein R₁₁ is —H, R₁₂ and R₁₄ is ═Cl, and R₇ is selected from —H, ═O and ═N—O—R₁, where R₁ is selected from —H, —CH₃, and —CH₂—CH═CH₂;
I) dehydroabietic acid derivatives according to formula I and all stereoisomers thereof, wherein R₁₁, R₁₂ and R₁₄ is ═Cl, and R₇ is selected from —H, ═O, and ═N—O—R₁, where R₁ is selected from —H, —CH₃, and —CH₂—CH═CH₂; and
m) dehydroabietic acid derivative according to formula I and all stereoisomers thereof wherein R₁₁, R₁₂ and R₁₄ is —H, and R₇ is selected from —H, ═O, —OH, and ═N—O—R₁, where R₁ is selected from —H, —CH₃, and —CH₂—CH═CH₂.

3. Dehydroabietic acid derivatives for use according claim 1, wherein Rug, R₁₂, and R₁₄ are independently selected from hydrogen and halogen, R₃ is isopropyl; and
R₇ is —H, ═N—O— CH₃ or ═N—O—CH₂—CH═CH₂ with the proviso that R₁₂ is not Br.

4. Dehydroabietic acid derivatives for use according to claim 2, selected from the group of:
(1R,4aS,E)-9-((allyloxy)imino)-6-chloro-7-isopropyl-1,4a-dimethyl-1,2,3,4,4a,9,10,10a-octahydrophenanthrene-1-carboxylic acid;
1R,4aS,E)-6-fluoro-7-isopropyl-9-(methoxyimino)-1,4a-dimethyl-1,2,3,4,4a,9,10,10a-octahydrophenanthrene-1-carboxylic acid;
(1R,4aS,E)-6-chloro-7-isopropyl-9-methoxyimino-1,4a-dimethyl-1,2,3,4,4a,9,10,10a-octahydrophenanthrene-1-carboxylic acid;
(1R,4aS,10aR)-6,8-dichloro-7-isopropyl-9-(methoxyimino)-1,4a-dimethyl-1,2,3,4,4a,9,10,10a-octahydrophenanthrene-1-carboxylic acid;
(1R,4aS,E)-9-((allyloxy)imino)-6-fluoro-7-isopropyl-1,4a-dimethyl-1,2,3,4,4a,9,10,10a-octahydrophenanthrene-1-carboxylic acid;
(1R,4aS,E)-9-((allyloxy)imino)-8-chloro-7-isopropyl-1,4a-dimethyl-1,2,3,4,4a,9,10,10a-octahydrophenanthrene-1-carboxylic acid;
(1R,4aS,E)-8-chloro-7-isopropyl-9-methoxyimino-1,4a-dimethyl-1,2,3,4,4a,9,10,10a-octahydrophenanthrene-1-carboxylic acid;
(1R,4aS)-8-chloro-7-isopropyl-1,4a-dimethyl-1,2,3,4,4a,9,10,10a-octahydrophenanthrene-1-carboxylic acid;
(1R,4aS)-6-chloro-7-isopropyl-1,4a-dimethyl-1,2,3,4,4a,9,10,10a-octahydrophenanthrene-1-carboxylic acid;
(1R,4aS)-8-bromo-7-isopropyl-1,4a-dimethyl-1,2,3,4,4a,9,10,10a-octahydrophenanthrene-1-carboxylic acid;
(1R,4aS,E)-6-iodo-7-isopropyl-1,4a-dimethyl-1,2,3,4,4a,9,10,10a-octahydrophenanthrene-1-carboxylic acid;
1R,4aS)-5-chloro-7-isopropyl-9-(methoxyimino)-1,4a-dimethyl-1,2,3,4,4a,9,10,10a-octahydrophenanthrene-1-carboxylic acid;
(1R,4aS,E)-9-((allyloxy)imino)-6-iodo-7-isopropyl-1,4a-dimethyl-1,2,3,4,4a,9,10,10a-octahydrophenanthrene-1-carboxylic acid;
(1R,4aS,E)-6-iodo-7-isopropyl-9-(methoxyimino)-1,4a-dimethyl-1,2,3,4,4a,9,10,10a-octahydrophenanthrene-1-carboxylic acid;
(1R,4aS,E)-9-(hydroxyimino)-6-iodo-7-isopropyl-1,4a-dimethyl-1,2,3,4,4a,9,10,10a-octahydrophenanthrene-1-carboxylic acid;
(1R,4aS,10aR)−)-8-iodo-7-isopropyl-9-methoxyimino-1,4a-dimethyl-1,2,3,4,4a,9,10,10a-octahydrophenanthrene-1-carboxylic acid; and
(1R,4aS,E)-8-iodo-7-isopropyl-1,4a-dimethyl-1,2,3,4,4a,9,10,10a-octahydrophenanthrene-1-carboxylic acid.

5. Dehydroabietic acid derivatives for use according to claim 1, wherein R₇ is —H, ═N—O—CH₃ or ═N—O—CH₂—CH═CH₂; R₃ is isopropyl; and R₁₁, R₁₂ and R₁₄ independently are selected from —H, —F, —Cl, —Br.

6. Dehydroabietic acid derivatives for use according to claim 5selected from the group of:
(1R,4aS,E)-6-bromo-7-isopropyl-9-(methoxyimino)-1,4a-dimethyl-1,2,3,4,4a,9,10,10a-octahydrophenanthrene-1-carboxylic acid;
(1R,4aS,E)-6-bromo-9-((allyloxy)imino)-7-isopropyl-1,4a-dimethyl-1,2,3,4,4a,9,10,10a-octahydrophenanthrene-1-carboxylic acid; and
(1R,4aS,E)-6-bromo-9-(hydroxyimino)-7-isopropyl-1,4a-dimethyl-1,2,3,4,4a,9,10,10a-octahydrophenanthrene-1-carboxylic acid.

7. Dehydroabietic acid derivatives for use according to claim 1, wherein R₇ is ═O, ═N—O—CH₃ or ═N—O—CH₂—CH═CH₂; R₃ is isopropyl; and R₁₁, R₁₂ and R₁₄ independently are selected from—hydrogen and halogen, with the proviso that R₁₂ is not bromo.

8. Dehydroabietic acid derivatives for use according to claim 7 selected from the group of:
(1R,4aS,E)-6-iodo-7-isopropyl-9-oxo-1,4a-dimethyl-1,2,3,4,4a,9,10,10a-octahydrophenanthrene-1-carboxylic acid;
(1R,4aS,E)-6-iodo-7-isopropyl-9-(methoxyimino)-1,4a-dimethyl-1,2,3,4,4a,9,10,10a-octahydrophenanthrene-1-carboxylic acid;
(1R,4aS,E)-8-iodo-7-isopropyl-9-oxo-1,4a-dimethyl-1,2,3,4,4a,9,10,10a-octahydrophenanthrene-1-carboxylic acid; and
(1R,4aS,E)-9-(hydroxyimino)-6-iodo-7-isopropyl-1,4a-dimethyl-1,2,3,4,4a,9,10,10a-octahydrophenanthrene-1-carboxylic acid.

9. Dehydroabietic acid derivatives for use according to claim 1, wherein R₇ is selected from hydrogen, halogen, and ═N—O—R₁ and where R₁₃ is selected from H or halogen.

10. Dehydroabietic acid derivatives for use according to claim 9 selected from the group of:
(1R,4aS)-6,7,8-trichloro-1,4a-dimethyl-1,2,3,4,4a,9,10,10a-octahydrophenanthrene-1-carboxylic acid;
(1R,4aS)-7,8-dichloro-1,4a-dimethyl-1,2,3,4,4a,9,10,10a-octahydrophenanthrene-1-carboxylic acid; and
(1R,4aS)-5,6,7,8-tetrachloro-1,4a-dimethyl-1,2,3,4,4a,9,10,10a-octahydrophenanthrene-1-carboxylic acid.

11. Dehydroabietic acid derivatives for use according to claim 1, wherein R₁₁ and R₁₄ are hydrogen, R₁₂ is R₂, R₁₃ is R₃, and R₇═N—O—R₁.

12. Dehydroabietic acid derivatives for use according to claim 11, selected from the group of:
(1R,4aS,E)-9-(methoxyimino)-7-isopropyl-1,4a-dimethyl-6-vinyl-1,2,3,4,4a,9,10,10a-octahydrophenanthrene-1-carboxylic acid;
(1R,4aS,E)-9-((allyloxy)imino)-7-isopropyl-1,4a-dimethyl-6-vinyl-1,2,3,4,4a,9,10,10a-octahydrophenanthrene-1-carboxylic acid;
(1R,4aS,E)-6-cyclopropyl-7-isopropyl-9-(methoxyimino)-1,4a-dimethyl-1,2,3,4,4a,9,10,10a-octahydrophenanthrene-1-carboxylic acid; and
(1R,4aS,E)-9-((allyloxy)imino)-6-cyclopropyl-7-isopropyl-1,4a-dimethyl-1,2,3,4,4a,9,10,10a-octahydrophenanthrene-1-carboxylic acid.

13. Dehydroabietic acid derivatives for use according to claim 1, wherein R₁₁ is hydrogen, R₁₂ and R₁₄ is selected from hydrogen and halogen R₃ is isopropyl, and R₇ is carbonyl.

14. Dehydroabietic acid derivatives for use according to claim 1 selected from the compounds included in the groups a) to m):
a) dehydroabietic acid derivatives according to formula I and all stereoisomers thereof, wherein R₁₂ is —F, R₁₁ and R₁₄ is —H, R₁₃ is isopropyl, and R₇ is selected from —H, ═O and ═N—O—R₁, where R₁ is selected from —H, —CH₃, —CH₂—CH═CH₂, and —CH₂—C₆H₅;
b) dehydroabietic acid derivatives according to formula I and all stereoisomers thereof, wherein R₁₁ and R₁₂ is —H, R₁₄ is —F, R₁₃ is isopropyl, and R₇ is selected from —H, ═O and ═N—O—R₁, where R₁ is selected from —CH₃, —CH₂—CH═CH₂, and —CH₂—C₆H₅;
c) dehydroabietic acid derivatives according to formula I and all stereoisomers thereof, wherein R₁₂ is —Cl, R₁₁ and R₁₄ is —H, R₁₃ is isopropyl, and R₇ is selected from —H, ═O and ═N—O—R₁, where R₁ is selected from —H, —CH₃, —CH₂—CH═CH₂, and —CH₂—C₆H₅;
d) dehydroabietic acid derivatives according to formula I and all stereoisomers thereof, wherein R₁₁ and R₁₂ is —H, R₁₃ is isopropyl, R₁₄ is —C1, and R₇ is selected from —H, ═O and ═N—O—R₁, where R₁ is selected from —H, —CH₃, —CH₂—CH═CH₂, and —CH₂—C₆H₅;
e) dehydroabietic acid derivatives according to formula I and all stereoisomers thereof, wherein R₁₁ is ═Cl, R₁₃ is isopropyl, R₁₂ and R₁₄ are —H, R₇ is selected from —H, ═O and ═N—O—R₁, where R₁ is selected from —H, —CH₃, —CH₂—CH═CH₂, and —CH₂—C₆H₅;
f) dehydroabietic acid derivatives according to formula I and all stereoisomers thereof, wherein R₁₁ and R₁₄ is —H, R₁₂ is —Br, R₁₃ is isopropyl, and R₇ is selected from ═O and ═N—O—CH₂—C₆H₅;
g) dehydroabietic acid derivatives according to formula I and all stereoisomers thereof, wherein R₁₁ and R₁₂ is —H, R₁₃ is isopropyl, R₁₄ is —Br, and R₇ is selected from ═O and ═N—O—R₁, where R₁ is selected from —H, —CH₂—CH═CH₂, and —CH₂—C₆H₅;
h) dehydroabietic acid derivatives according to formula I and all stereoisomers thereof, wherein R₁₂ is R₁₁ and R₁₄ is —H, R₁₃ is isopropyl, and R₇ is selected from —H, ═O and ═N—O—R₁, where R₁ is selected from —H, —CH₃, —CH₂—CH═CH₂, and —CH₂—C₆H₅;
i) dehydroabietic acid derivatives according to formula I and all stereoisomers thereof, wherein R₁₁ and R₁₂ is —H, R₁₃ is isopropyl, R₁₄ is —I, and R₇ is selected from ═O and ═N—O—R₁, where R₁ is selected from —H, —CH₃, —CH₂—CH═CH₂, and —CH₂—C₆H₅;
j) dehydroabietic acid derivatives according to formula I and all stereoisomers thereof, wherein R₁₁, R₁₂ and R₁₄ is —Cl, R₁₃ is isopropyl, and R₇ is selected from —H, ═O, and ═N—O—CH₂—C₆H₅;
k) dehydroabietic acid derivatives according to formula I and all stereoisomers thereof, wherein R₁₁ and R₁₂ are independently selected from hydrogen and halogen, R₇ is selected from hydrogen, halogen and ═N—O—R₁, wherein R₁ is selected from —H, —CH₃, —CH₂—CH═CH₂, and —CH₂—C₆H₅ and wherein R₁₃ is selected from H or halogen;
I) dehydroabietic acid derivatives according to formula I and all stereoisomers thereof, wherein R₁₁ and R₁₄ are hydrogen R₁₂ is a straight, branched or cyclic saturated or unsaturated hydrocarbon comprising from 1 to 6 carbon atoms (C1-C6 alkyl, C2-C6 alkenyl and C3-C6 cycloalkyl; said alkyl, alkenyl, and cycloalkyl optionally being substituted with at least one halogen), R₁₃ is isopropyl, and R₇═N—O—R₁, R₁ is selected from —H, —CH₃, and —CH₂—CH═CH₂, and —CH₂—C₆H₅; and
m) dehydroabietic acid derivatives according to formula I and all stereoisomers thereof, wherein R₁₁ is hydrogen, R₁₂ and R₁₄ is selected from hydrogen and iodo, R₃ is isopropyl, and R₇ is ═O.

15. Dehydroabietic acid derivatives from groups a) to m) of claim 14 selected from:
(1R,4aS)-8-chloro-7-isopropyl-1,4a-dimethyl-1,2,3,4,4a,9,10,10a-octahydrophenanthrene-1-carboxylic acid;
(1R,4aS)-6-chloro-7-isopropyl-1,4a-dimethyl-1,2,3,4,4a,9,10,10a-octahydrophenanthrene-1-carboxylic acid;
(1R,4aS,E)-8-chloro-7-isopropyl-9-methoxyimino-1,4a-dimethyl-1,2,3,4,4a,9,10,10a-octahydrophenanthrene-1-carboxylic acid;
(1R,4aS,E)-9-((allyloxy)imino)-8-chloro-7-isopropyl-1,4a-dimethyl-1,2,3,4,4a,9,10,10a-octahydrophenanthrene-1-carboxylic acid;
(1R,4aS,E)-6-chloro-7-isopropyl-9-oxo-1,4a-dimethyl-1,2,3,4,4a,9,10,10a-octahydrophenanthrene-1-carboxylic acid;
(1R,4aS,E)-6-chloro-7-isopropyl-9-hydroxyimino-1,4a-dimethyl-1,2,3,4,4a,9,10,10a-octahydrophenanthrene-1-carboxylic acid;
(1R,4aS,E)-9-((allyloxy)imino)-6-chloro-7-isopropyl-1,4a-dimethyl-1,2,3,4,4a,9,10,10a-octahydrophenanthrene-1-carboxylic acid;
(1R,4aS,E)-6-bromo-7-isopropyl-1,4a-dimethyl-1,2,3,4,4a,9,10,10a-octahydrophenanthrene-1-carboxylic acid;
(1R,4aS)-8-bromo-7-isopropyl-1,4a-dimethyl-1,2,3,4,4a,9,10,10a-octahydrophenanthrene-1-carboxylic acid;
(1R,4aS,E)-9-((allyloxy)imino)-6-fluoro-7-isopropyl-1,4a-dimethyl-1,2,3,4,4a,9,10,10a-octahydrophenanthrene-1-carboxylic acid;
(1R,4aS,E)-6-fluoro-7-isopropyl-9-(methoxyimino)-1,4a-dimethyl-1,2,3,4,4a,9,10,10a-octahydrophenanthrene-1-carboxylic acid;
(1R,4aS,E)-6-iodo-7-isopropyl-1,4a-dimethyl-1,2,3,4,4a,9,10,10a-octahydrophenanthrene-1-carboxylic acid;
(1R,4aS,E)-9-((allyloxy)imino)-6-iodo-7-isopropyl-1,4a-dimethyl-1,2,3,4,4a,9,10,10a-octahydrophenanthrene-1-carboxylic acid;
(1R,4aS,E)-6-iodo-7-isopropyl-9-(methoxyimino)-1,4a-dimethyl-1,2,3,4,4a,9,10,10a-octahydrophenanthrene-1-carboxylic acid;
(1R,4aS,E)-6-chloro-7-isopropyl-9-methoxyimino-1,4a-dimethyl-1,2,3,4,4a,9,10,10a-octahydrophenanthrene-1-carboxylic acid;
(1R,4aS)-5-chloro-7-isopropyl-9-(methoxyimino)-1,4a-dimethyl-1,2,3,4,4a,9,10,10a-octahydrophenanthrene-1-carboxylic acid, (1R,4aS)-6,7,8-trichloro-1,4a-dimethyl-1,2,3,4,4a,9,10,10a-octahydrophenanthrene-1-carboxylic acid;
(1R,4aS)-7,8-dichloro-1,4a-dimethyl-1,2,3,4,4a,9,10,10a-octahydrophenanthrene-1-carboxylic acid;
(1R,4aS)-5,6,7,8-tetrachloro-1,4a-dimethyl-1,2,3,4,4a,9,10,10a-octahydrophenanthrene-1-carboxylic acid;
(1R,4aS,E)-9-(methoxyimino)-7-isopropyl-1,4a-dimethyl-6-vinyl-1,2,3,4,4a,9,10,10a-octahydrophenanthrene-1-carboxylic acid;
(1R,4aS,E)-9-((allyloxy)imino)-7-isopropyl-1,4a-dimethyl-6-vinyl-1,2,3,4,4a,9,10,10a-octahydrophenanthrene-1-carboxylic acid;
(1R,4aS,E)-6-cyclopropyl-7-isopropyl-9-(methoxyimino)-1,4a-dimethyl-1,2,3,4,4a,9,10,10a-octahydrophenanthrene-1-carboxylic acid;
(1R,4aS,E)-9-((allyloxy)imino)-6-cyclopropyl-7-isopropyl-1,4a-dimethyl-1,2,3,4,4a,9,10,10a-octahydrophenanthrene-1-carboxylic acid;
(1R,4aS,E)-8-iodo-7-isopropyl-1,4a-dimethyl-1,2,3,4,4a,9,10,10a-octahydrophenanthrene-1-carboxylic acid;
(1R,4aS,E)-9-(hydroxyimino)-6-iodo-7-isopropyl-1,4a-dimethyl-1,2,3,4,4a,9,10,10a-octahydrophenanthrene-1-carboxylic acid;
(1R,4aS,10aR)+8-iodo-7-isopropyl-9-methoxyimino-1,4a-dimethyl-1,2,3,4,4a,9,10,10a-octahydrophenanthrene-1-carboxylic acid;
(1 S,4aS)-1,4a-dimethyl-1,2,3,4,4a,9,10,10a-octahydrophenanthrene-1-carboxylic acid;
(1 S,4aS)-6-chloro-1,4a-dimethyl-1,2,3,4,4a,9,10,10a-octahydrophenanthrene-1-carboxylic acid; and
(1 S,4aS)-7-chloro-1,4a-dimethyl-1,2,3,4,4a,9,10,10a-octahydrophenanthrene-1-carboxylic acid.
